(19) United States
(12) Patent Application Publication
(10) Pub. No.: US 2021/0038602 A1
(43) Pub. Date: Feb. 11, 2021

(54) COMBINATION TREATMENT OF ACUTE MYELOID LEUKEMIA

(71) Applicant: Boehringer Ingelheim International GmbH, Ingelheim am Rhein (DE)

(72) Inventors: Ulrike TONTSCH-GRUNT, Baden (AT); Anke BAUM, Moedling (AT); Dorothea Ingrid RUDOLPH, Vienna (AT)

(21) Appl. No.: 16/964,023

(22) PCT Filed: Jan. 24, 2019
(86) PCT No.: PCT/EP2019/051733

§ 371 Date: Jul. 22, 2020

(30) Foreign Application Priority Data
Jan. 25, 2018 (EP) 18153471.0

(51) Int. Cl. A61K 31/519; A61K 31/4188; A61K 31/5377; A61K 31/551; A61K 31/4985; A61K 31/501; A61K 31/55

(52) U.S. Cl. CPC A61K 31/519; A61K 31/4188; A61K 31/5377; A61K 31/55; A61K 31/4985; A61K 31/501; A61K 31/551

(57) ABSTRACT

The present invention relates to the use of volasertib, or a pharmaceutically acceptable salt thereof or a hydrate thereof, in combination with a BET inhibitor, or a pharmaceutically acceptable salt thereof or a hydrate thereof for treating patients suffering from acute myeloid leukemia (AML).

The present invention relates to the use of volasertib or a pharmaceutically acceptable salt thereof or the hydrate thereof in combination with a BET inhibitor or a pharmaceutically acceptable salt thereof or the hydrate thereof for treating patients suffering from acute myeloid leukemia (AML).

### BACKGROUND OF THE INVENTION

Acute myeloid leukemia (AML), also known as acute myelogenous leukemia, is a cancer of the myeloid line of blood cells, characterized by the rapid growth of abnormal white blood cells that accumulate in the bone marrow and interfere with the production of normal blood cells. As an acute leukemia, AML progresses rapidly and is typically fatal within weeks or months if left untreated. AML is the most prevalent form of adult acute leukemia, particularly among older adults and is slightly more common in men than women. There is an estimated prevalence of 30,000 cases of AML in the US and 47,000 in the EU.

The incidence of AML increases with age with a median age at diagnosis of 67 years. The global incidence CAGR for AML out to 2013 is 1.4%. An aging population, along with an increased incidence of treatment-related AML in cancer survivors, currently accounting for 10-20% of all AML cases, is expected to drive the incidence of AML. In addition, there is some geographic variation in the incidence of AML. In adults, the highest rates are seen in North America, Europe, and Oceania, while adult AML is less frequently diagnosed in Asia and Latin America.

AML accounts for approximately 1.2% of all cancer deaths. The 5 year survival rates for AML are low, driven by therapy failure and patients relapsing. Among patients <65 the 5 year survival rate is 34.4%, among patients >65 it is only 5%.

The WHO classification of myeloid neoplasms and acute leukemia is the current standard for classification of AML and incorporates genetic abnormalities into diagnostic algorithms. This classification is done by examining the appearance of the malignant cells under light microscopy and by using cytogenetics and molecular genetics to characterize any underlying chromosomal abnormalities or genetic changes. The subtypes impact on prognoses, responses to therapy and treatment decisions.

The efficacy of chemotherapeutic agents can be improved by using combination therapies with other compounds and/or improving the dosage schedule. Even if the concept of combining several therapeutic agents or improved dosage schedules already has been suggested, there is still a need for new and efficient therapeutic concepts for the treatment of cancer diseases, which show advantages over standard therapies.

Volasertib is a highly potent and selective inhibitor of the serine-threonine polo like kinase (PLK), a key regulator of cell-cycle progression. Volasertib is a second-generation dihydropteridinone derivative with distinct pharmacokinetic (PK) properties. The problem underlying this invention was to develop a combination treatment and improved dosage schedules for combination therapy of volasertib and a BET inhibitor in AML with maximal activity and limited toxicity.

Volasertib (I) is known as the compound N-[trans-4-[4-(cyclopropylmethyl)-1-piperazinyl]cyclohexyl]-4-[[(7R)-7-ethyl-5,6,7,8-tetrahydro-5-methyl-8-(1-methylethyl)-6-oxo-2-pteridinyl]amino]-3-methoxy-benzamide

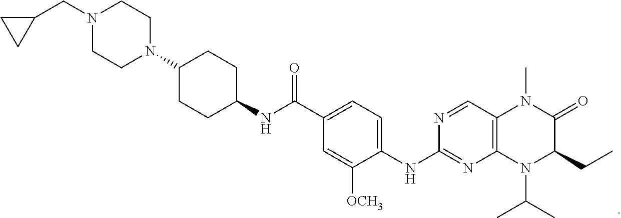

This compound is disclosed in WO 2004/076454. Furthermore, trihydrochloride salt forms and hydrates thereof are known from WO 2007/090844. They possess properties which make those forms especially suitable for pharmaceutical use. The above mentioned patent applications further disclose the use of this compound or its monoethanesulfonate salt for the preparation of pharmaceutical compositions intended especially for the treatment of diseases characterized by excessive or abnormal cell proliferation.

Document WO 2006/018182 discloses other combinations for the treatment of diseases involving cell proliferation.

BET inhibitors inhibit the binding of bromodomains to acetylated lysines on histone H3 and H4 and thus act as important regulators of gene transcription, and are useful for the treatment of AML. BET inhibitors belonging to different compound classes are known. WO 2014/076237 and WO 2014/076146, e.g., describe triazolopyrazine derivatives as BET inhibitors. WO 2014/068402 describes thienotriazolo diazepine derivatives as BET inhibitors. WO 2013/033268 describes further diazepine derivatives as BET inhibitors.

### SUMMARY OF THE INVENTION

In in-vitro experiments it has been found that the apoptotic effect resulting from the combined use of volasertib and a BET inhibitor is more effective than the effect resulting from the single use of each compound.

Accordingly, a first aspect of the present invention refers to a pharmaceutical combination comprising volasertib, or a pharmaceutically acceptable salt thereof or a hydrate thereof, and a BET inhibitor, or a pharmaceutically acceptable salt thereof or a hydrate thereof, for simultaneous, separate or sequential use of the active ingredients.

Another aspect of the present invention relates to a kit comprising a pharmaceutical composition comprising volasertib, or a pharmaceutically acceptable salt thereof or a hydrate thereof, and another pharmaceutical composition comprising a BET inhibitor, or a pharmaceutically acceptable salt thereof or a hydrate thereof.

Another aspect of the present invention relates to a pharmaceutical composition comprising a therapeutically effective amount of volasertib, or a pharmaceutically acceptable salt thereof or a hydrate thereof, and a therapeutically effective amount of a BET inhibitor, or a pharmaceutically acceptable salt thereof or a hydrate thereof.

Another aspect of the present invention relates to volasertib, or a pharmaceutically acceptable salt thereof or a hydrate thereof, for use in treating AML, characterized in that volasertib is administered in combination with a BET inhibitor, or a pharmaceutically acceptable salt thereof or a hydrate thereof, wherein both active ingredients are administered simultaneously, separately or sequentially.

Another aspect of the present invention relates to a BET inhibitor, or a pharmaceutically acceptable salt thereof or a hydrate thereof, for use in treating AML, characterized in that the BET inhibitor is administered in combination with volasertib, or a pharmaceutically acceptable salt thereof or a hydrate thereof, wherein both active ingredients are administered simultaneously, separately or sequentially.

Another aspect of the present invention relates to volasertib, or a pharmaceutically acceptable salt thereof or a hydrate thereof, for use in treating AML characterized in that volasertib is administered in combination with a BET inhibitor, or a pharmaceutically acceptable salt thereof or a hydrate thereof, according to a dosage schedule comprising or consisting of
a) administration of a therapeutically effective amount of volasertib, or a pharmaceutically acceptable salt thereof or a hydrate thereof, on at least one day during a treatment cycle and
b) administration of a therapeutically effective amount of a BET inhibitor, or a pharmaceutically acceptable salt thereof or a hydrate thereof, on at least one day of said treatment cycle to a patient suffering from AML.

Another aspect of the present invention relates to a method of treating AML comprising administering to a patient in need of such treatment a therapeutically effective amount of volasertib, or a pharmaceutically acceptable salt thereof or a hydrate thereof, in combination with a therapeutically effective amount of a BET inhibitor, or a pharmaceutically acceptable salt thereof or a hydrate thereof.

In one embodiment of all aspects of the invention disclosed herein the BET inhibitor, or a pharmaceutically acceptable salt thereof or a hydrate thereof, is a diazepine derivative.

In another embodiment of all aspects of the invention disclosed herein the BET inhibitor, or a pharmaceutically acceptable salt thereof or a hydrate thereof, is a triazolopyrazine derivative.

In another embodiment of all aspects of the invention disclosed herein the BET inhibitor, or a pharmaceutically acceptable salt thereof or a hydrate thereof, is a pyridinone derivative.

In another embodiment of all aspects of the invention disclosed herein the BET inhibitor, or a pharmaceutically acceptable salt thereof or a hydrate thereof, is selected from the compounds of table 1:

TABLE 1

| | |
|---|---|
| 1 | 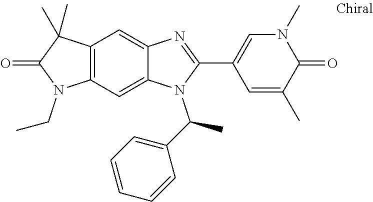 [SMILES: CCN1C(=O)C(C)(C)c2cc3nc(-c4cc(C)c(=O)n(C)c4)n([C@@H](C)c4ccccc4)c3cc21] |
| 2 | 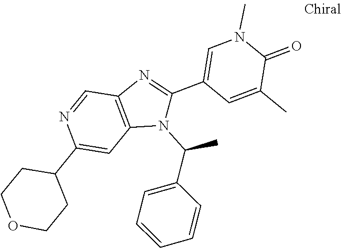 [SMILES: Cc1cc(-c2nc3cnc(C4CCOCC4)cc3n2[C@@H](C)c2ccccc2)cn(C)c1=O] |
| 3 | 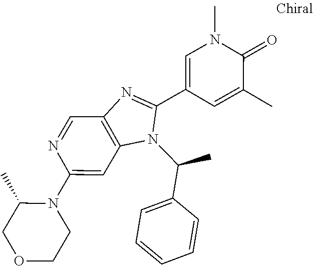 [SMILES: Cc1cc(-c2nc3cnc(N4CCOC[C@@H]4C)cc3n2[C@@H](C)c2ccccc2)cn(C)c1=O] |
| 4 | 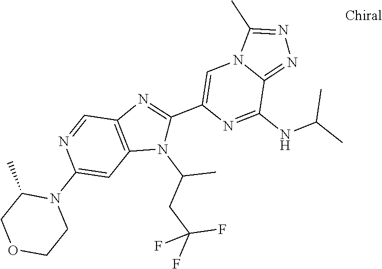 [SMILES: Cc1nnc2c(NC(C)C)nc(-c3nc4cnc(N5CCOC[C@@H]5C)cc4n3C(C)CC(F)(F)F)cn12] |
| 5 | 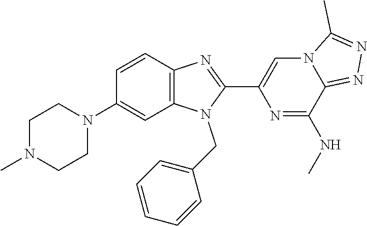 [SMILES: CNc1nc(-c2nc3ccc(N4CCN(C)CC4)cc3n2Cc2ccccc2)cn2c(C)nnc12] |
| 6 | 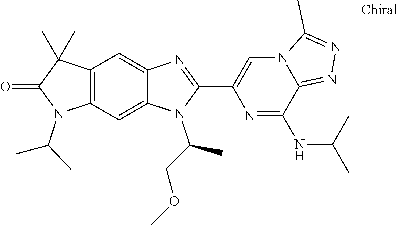 [SMILES: COC[C@H](C)n1c(-c2cn3c(C)nnc3c(NC(C)C)n2)nc2cc3c(cc21)N(C(C)C)C(=O)C3(C)C] |
| 7 | 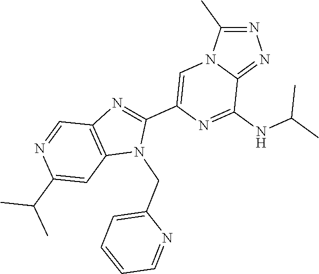 [SMILES: Cc1nnc2c(NC(C)C)nc(-c3nc4cnc(C(C)C)cc4n3Cc3ccccn3)cn12] |
| 8 | 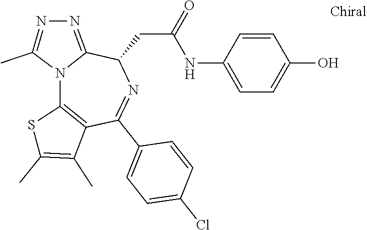 [SMILES: Cc1sc2c(c1C)C(c1ccc(Cl)cc1)=N[C@@H](CC(=O)Nc1ccc(O)cc1)c1nnc(C)n1-2] |
| 9 | 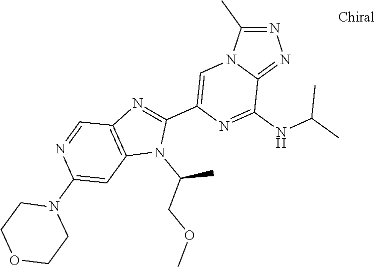 [SMILES: COC[C@H](C)n1c(-c2cn3c(C)nnc3c(NC(C)C)n2)nc2cnc(N3CCOCC3)cc21] |
| 10 | 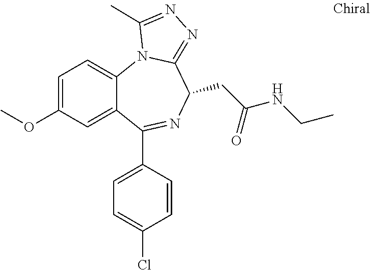 [SMILES: CCNC(=O)C[C@@H]1N=C(c2ccc(Cl)cc2)c2cc(OC)ccc2-n2c(C)nnc21] |
| 11 | 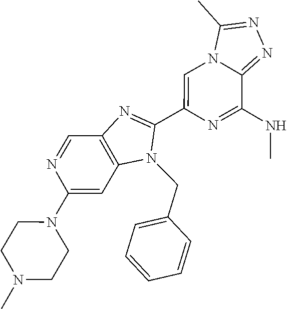 [SMILES: CNc1nc(-c2nc3cnc(N4CCN(C)CC4)cc3n2Cc2ccccc2)cn2c(C)nnc12] |
| 12 | 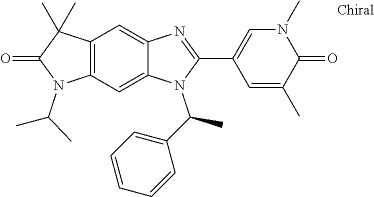 [SMILES: Cc1cc(-c2nc3cc4c(cc3n2[C@@H](C)c2ccccc2)N(C(C)C)C(=O)C4(C)C)cn(C)c1=O] |
| 13 | 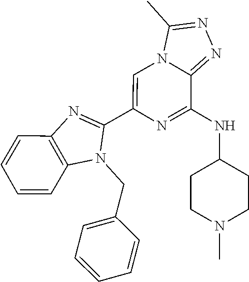 [SMILES: Cc1nnc2c(NC3CCN(C)CC3)nc(-c3nc4ccccc4n3Cc3ccccc3)cn12] |
| 14 | 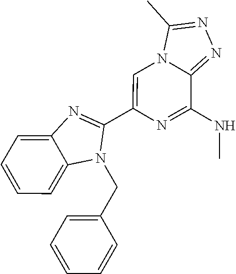 [SMILES: CNc1nc(-c2nc3ccccc3n2Cc2ccccc2)cn2c(C)nnc12] |
| 15 | 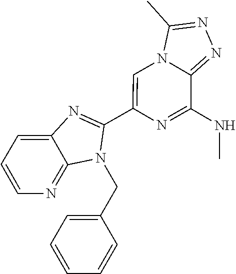 [SMILES: CNc1nc(-c2nc3cccnc3n2Cc2ccccc2)cn2c(C)nnc12] |
| 16 | 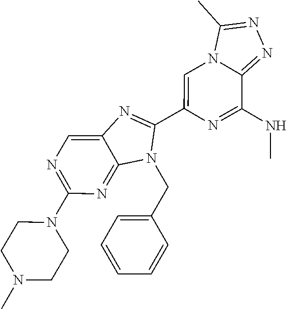 [SMILES: CNc1nc(-c2nc3cnc(N4CCN(C)CC4)nc3n2Cc2ccccc2)cn2c(C)nnc12] |
| 17 | 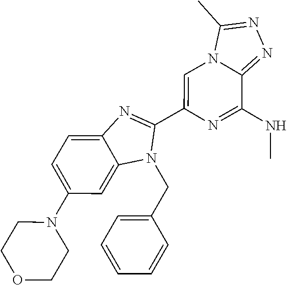 [SMILES: CNc1nc(-c2nc3ccc(N4CCOCC4)cc3n2Cc2ccccc2)cn2c(C)nnc12] |
| 18 | 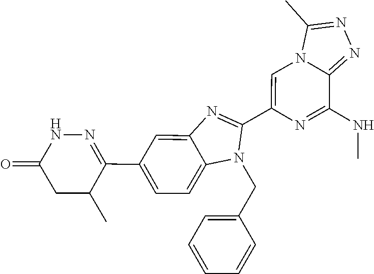 [SMILES: CNc1nc(-c2nc3cc(C4=NNC(=O)CC4C)ccc3n2Cc2ccccc2)cn2c(C)nnc12] |
| 19 | 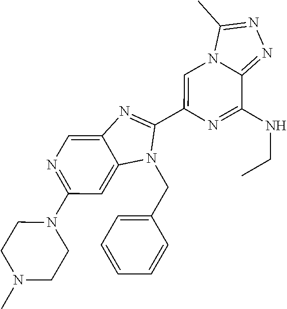 [SMILES: CCNc1nc(-c2nc3cnc(N4CCN(C)CC4)cc3n2Cc2ccccc2)cn2c(C)nnc12] |
| 20 | 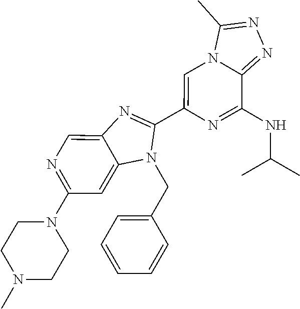 [SMILES: Cc1nnc2c(NC(C)C)nc(-c3nc4cnc(N5CCN(C)CC5)cc4n3Cc3ccccc3)cn12] |
| 21 | 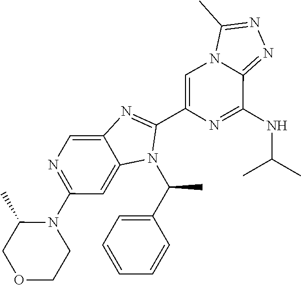 [SMILES: Cc1nnc2c(NC(C)C)nc(-c3nc4cnc(N5CCOC[C@@H]5C)cc4n3[C@@H](C)c3ccccc3)cn12] |
| 22 | 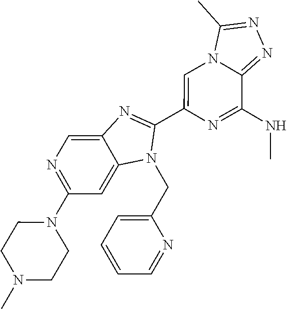 [SMILES: CNc1nc(-c2nc3cnc(N4CCN(C)CC4)cc3n2Cc2ccccn2)cn2c(C)nnc12] |
| 23 | 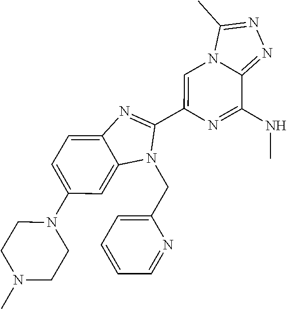 [SMILES: CNc1nc(-c2nc3ccc(N4CCN(C)CC4)cc3n2Cc2ccccn2)cn2c(C)nnc12] |
| 24 | 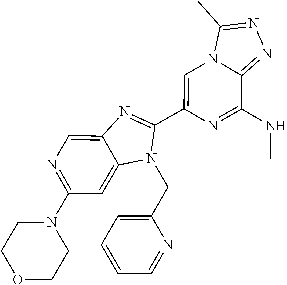 [SMILES: CNc1nc(-c2nc3cnc(N4CCOCC4)cc3n2Cc2ccccn2)cn2c(C)nnc12] |
| 25 | 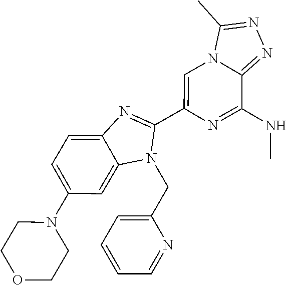 [SMILES: CNc1nc(-c2nc3ccc(N4CCOCC4)cc3n2Cc2ccccn2)cn2c(C)nnc12] |
| 26 | 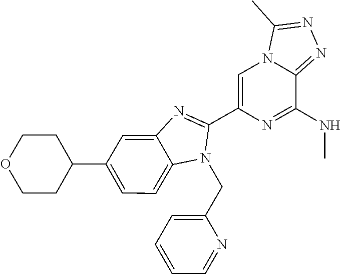 [SMILES: CNc1nc(-c2nc3cc(C4CCOCC4)ccc3n2Cc2ccccn2)cn2c(C)nnc12] |
| 27 | 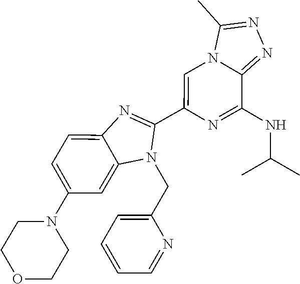 [SMILES: Cc1nnc2c(NC(C)C)nc(-c3nc4ccc(N5CCOCC5)cc4n3Cc3ccccn3)cn12] |
| 28 | 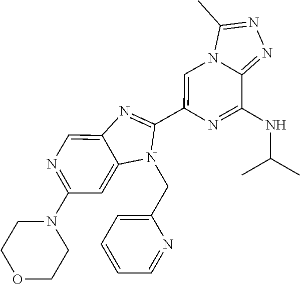 [SMILES: Cc1nnc2c(NC(C)C)nc(-c3nc4cnc(N5CCOCC5)cc4n3Cc3ccccn3)cn12] |
| 29 | 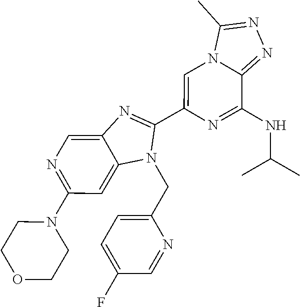 [SMILES: Cc1nnc2c(NC(C)C)nc(-c3nc4cnc(N5CCOCC5)cc4n3Cc3ccc(F)cn3)cn12] |
| 30 | 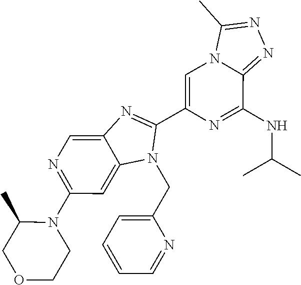 [SMILES: Cc1nnc2c(NC(C)C)nc(-c3nc4cnc(N5CCOC[C@H]5C)cc4n3Cc3ccccn3)cn12] |
| 31 | 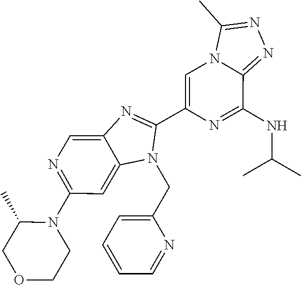 [SMILES: Cc1nnc2c(NC(C)C)nc(-c3nc4cnc(N5CCOC[C@@H]5C)cc4n3Cc3ccccn3)cn12] |
| 32 | 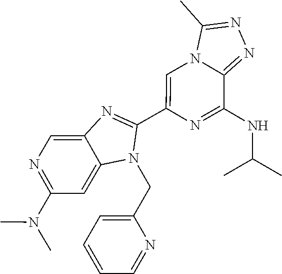 [SMILES: Cc1nnc2c(NC(C)C)nc(-c3nc4cnc(N(C)C)cc4n3Cc3ccccn3)cn12] |
| 33 | 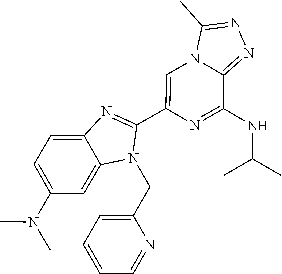 [SMILES: Cc1nnc2c(NC(C)C)nc(-c3nc4ccc(N(C)C)cc4n3Cc3ccccn3)cn12] |
| 34 | 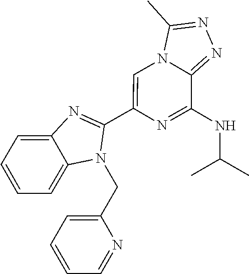 [SMILES: Cc1nnc2c(NC(C)C)nc(-c3nc4ccccc4n3Cc3ccccn3)cn12] |
| 35 | 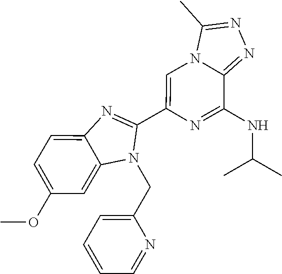 [SMILES: COc1ccc2nc(-c3cn4c(C)nnc4c(NC(C)C)n3)n(Cc3ccccn3)c2c1] |
| 36 | 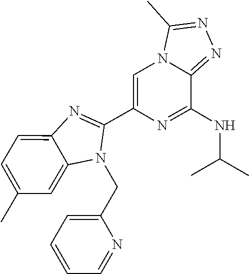 [SMILES: Cc1ccc2nc(-c3cn4c(C)nnc4c(NC(C)C)n3)n(Cc3ccccn3)c2c1] |
| 37 | 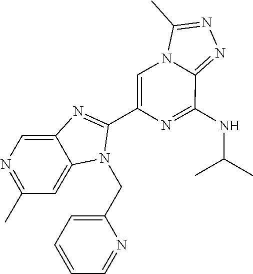 [SMILES: Cc1cc2c(cn1)nc(-c1cn3c(C)nnc3c(NC(C)C)n1)n2Cc1ccccn1] |
| 38 | 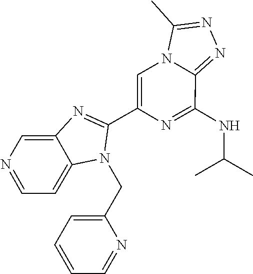 [SMILES: Cc1nnc2c(NC(C)C)nc(-c3nc4cnccc4n3Cc3ccccn3)cn12] |
| 39 | 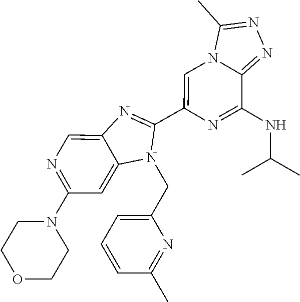 [SMILES: Cc1cccc(Cn2c(-c3cn4c(C)nnc4c(NC(C)C)n3)nc3cnc(N4CCOCC4)cc32)n1] |
| 40 | 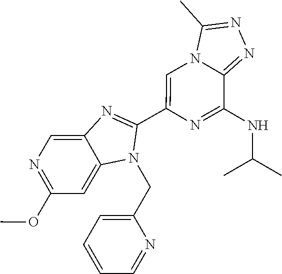 [SMILES: COc1cc2c(cn1)nc(-c1cn3c(C)nnc3c(NC(C)C)n1)n2Cc1ccccn1] |
| 41 | 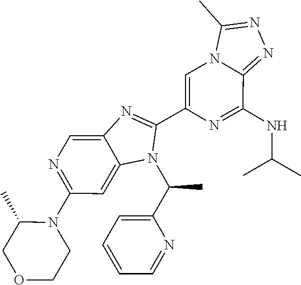 [SMILES: Cc1nnc2c(NC(C)C)nc(-c3nc4cnc(N5CCOC[C@@H]5C)cc4n3[C@@H](C)c3ccccn3)cn12] |
| 42 | 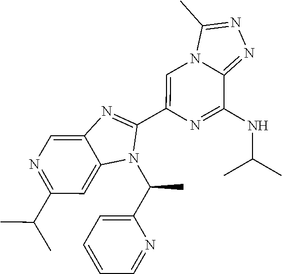 [SMILES: Cc1nnc2c(NC(C)C)nc(-c3nc4cnc(C(C)C)cc4n3[C@@H](C)c3ccccn3)cn12] |
| 43 | 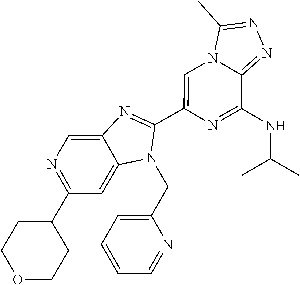 [SMILES: Cc1nnc2c(NC(C)C)nc(-c3nc4cnc(C5CCOCC5)cc4n3Cc3ccccn3)cn12] |
| 44 | 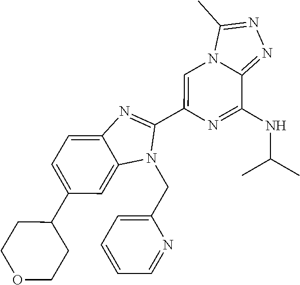 [SMILES: Cc1nnc2c(NC(C)C)nc(-c3nc4ccc(C5CCOCC5)cc4n3Cc3ccccn3)cn12] |
| 45 | 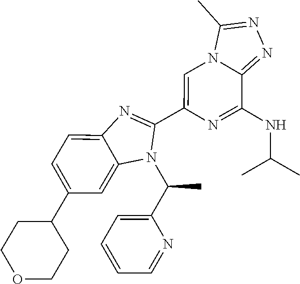 [SMILES: Cc1nnc2c(NC(C)C)nc(-c3nc4ccc(C5CCOCC5)cc4n3[C@@H](C)c3ccccn3)cn12] |
| 46 | 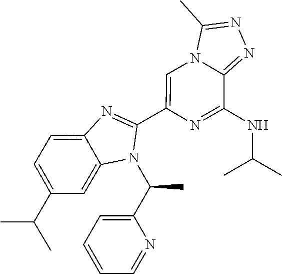 [SMILES: Cc1nnc2c(NC(C)C)nc(-c3nc4ccc(C(C)C)cc4n3[C@@H](C)c3ccccn3)cn12] |
| 47 | 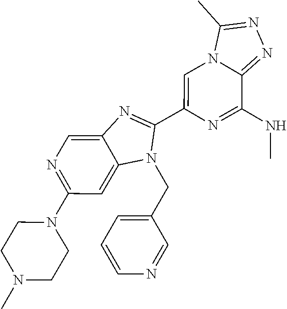 [SMILES: CNc1nc(-c2nc3cnc(N4CCN(C)CC4)cc3n2Cc2cccnc2)cn2c(C)nnc12] |
| 48 | 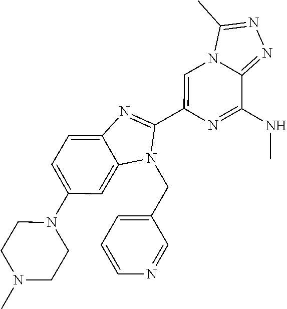 [SMILES: CNc1nc(-c2nc3ccc(N4CCN(C)CC4)cc3n2Cc2cccnc2)cn2c(C)nnc12] |
| 49 | 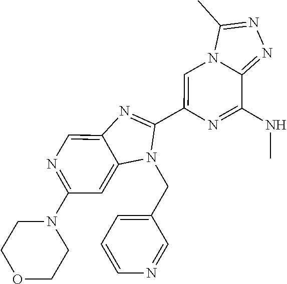 [SMILES: CNc1nc(-c2nc3cnc(N4CCOCC4)cc3n2Cc2cccnc2)cn2c(C)nnc12] |
| 50 | 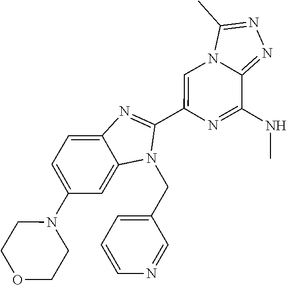 [SMILES: CNc1nc(-c2nc3ccc(N4CCOCC4)cc3n2Cc2cccnc2)cn2c(C)nnc12] |
| 51 | 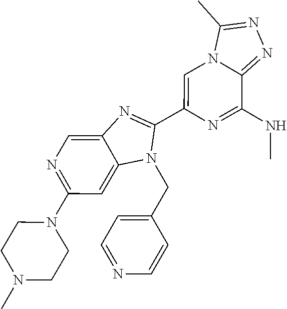 [SMILES: CNc1nc(-c2nc3cnc(N4CCN(C)CC4)cc3n2Cc2ccncc2)cn2c(C)nnc12] |
| 52 | 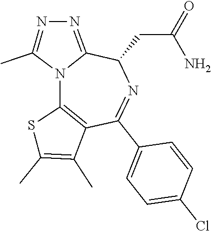 [SMILES: Cc1sc2c(c1C)C(c1ccc(Cl)cc1)=N[C@@H](CC(N)=O)c1nnc(C)n1-2] |
| 53 | 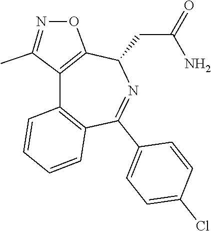 [SMILES: Cc1noc2c1-c1ccccc1C(c1ccc(Cl)cc1)=N[C@H]2CC(N)=O] |

### BRIEF DESCRIPTION OF THE FIGURES

FIGS. 1-12

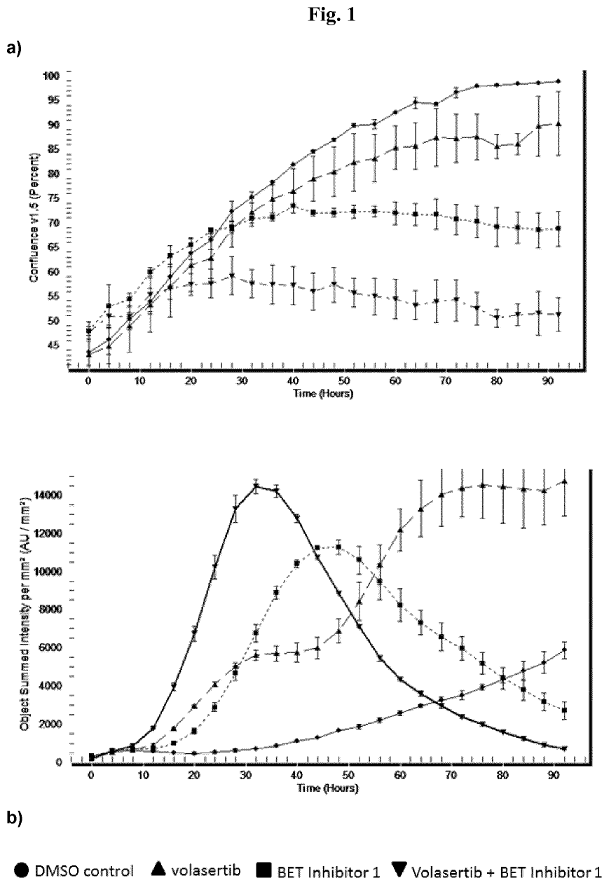

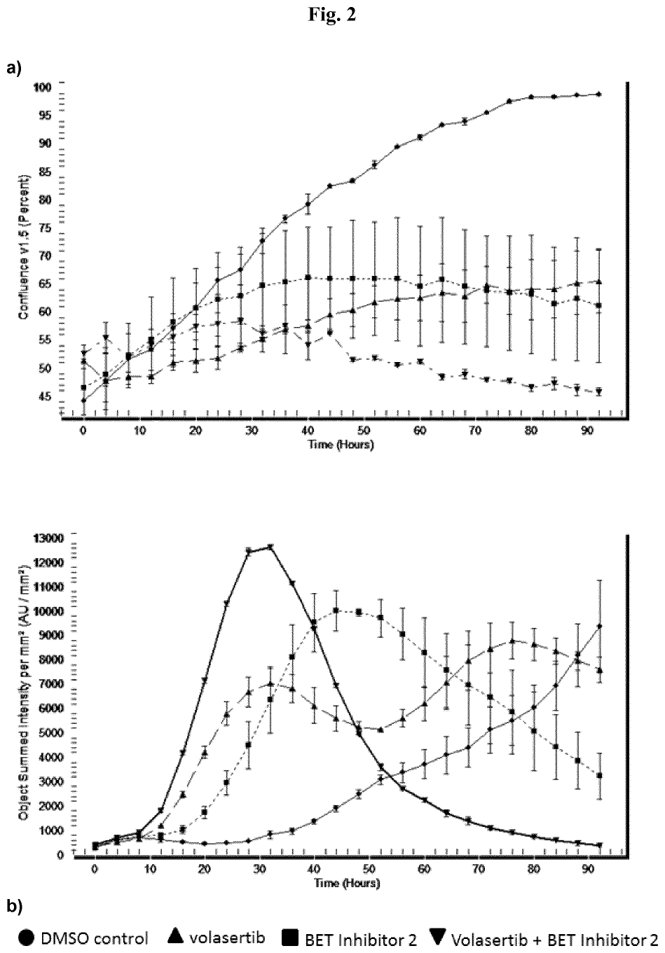

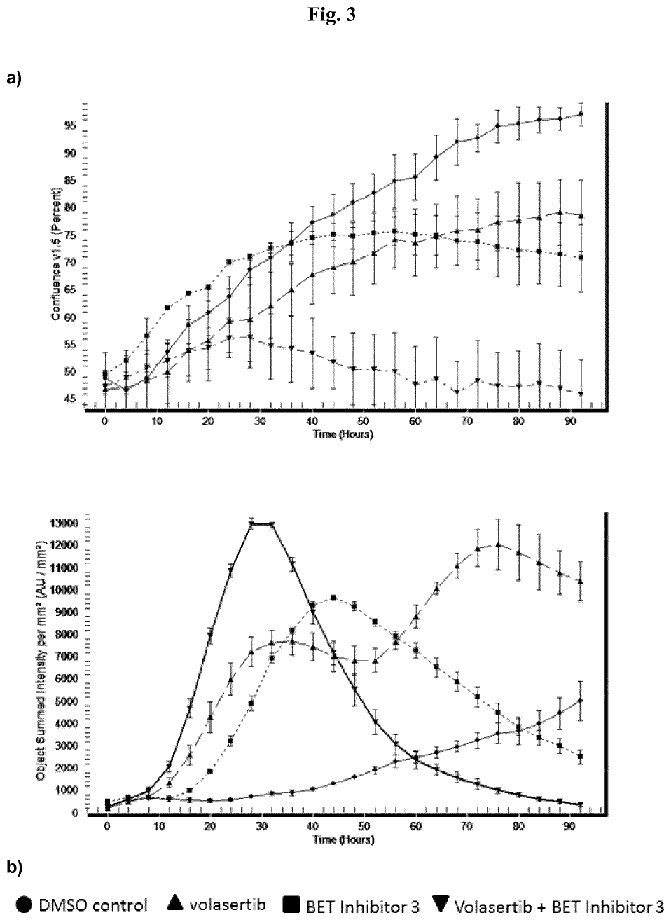

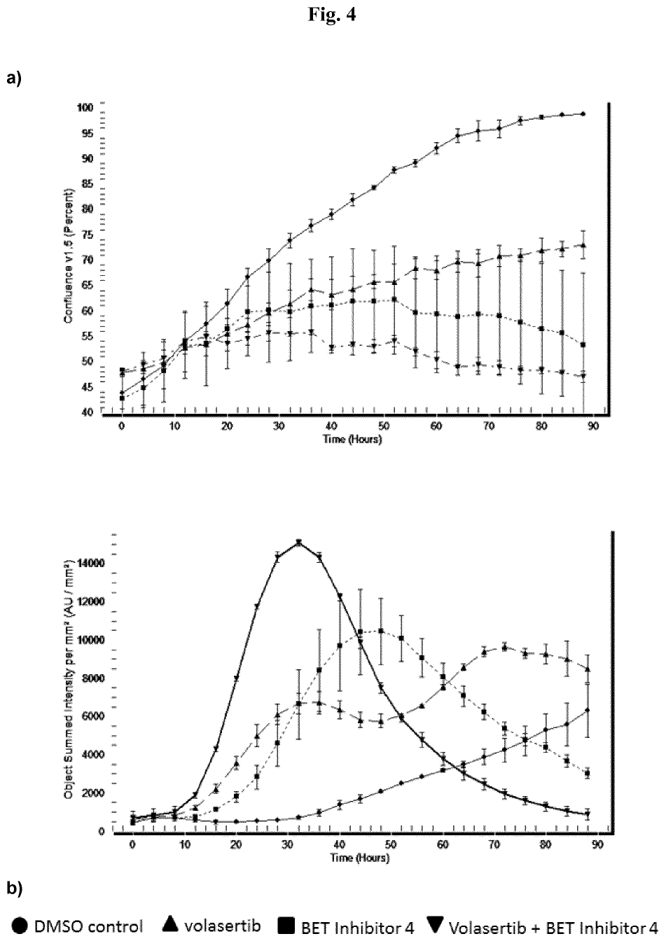

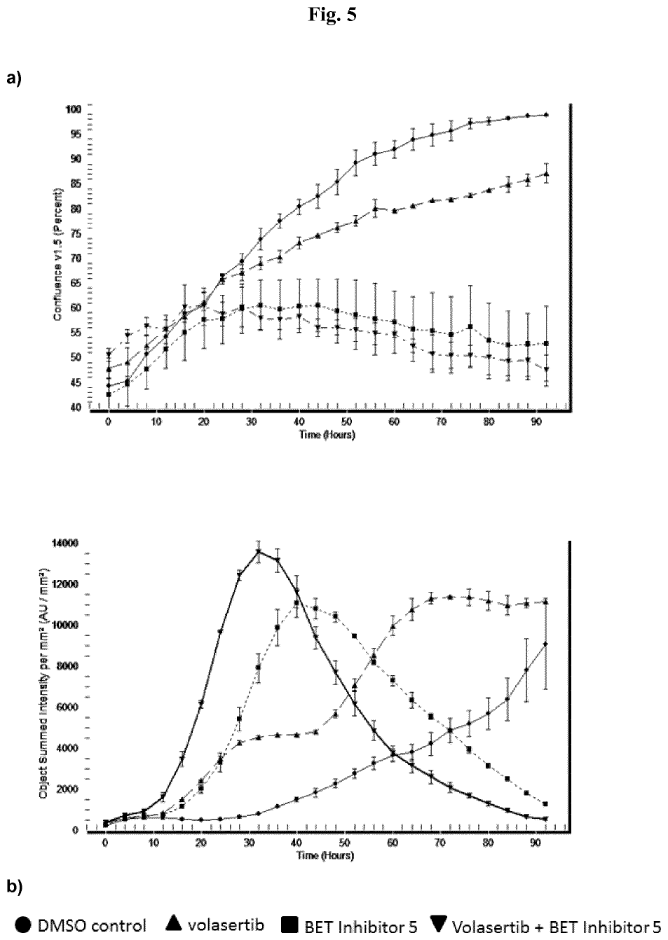

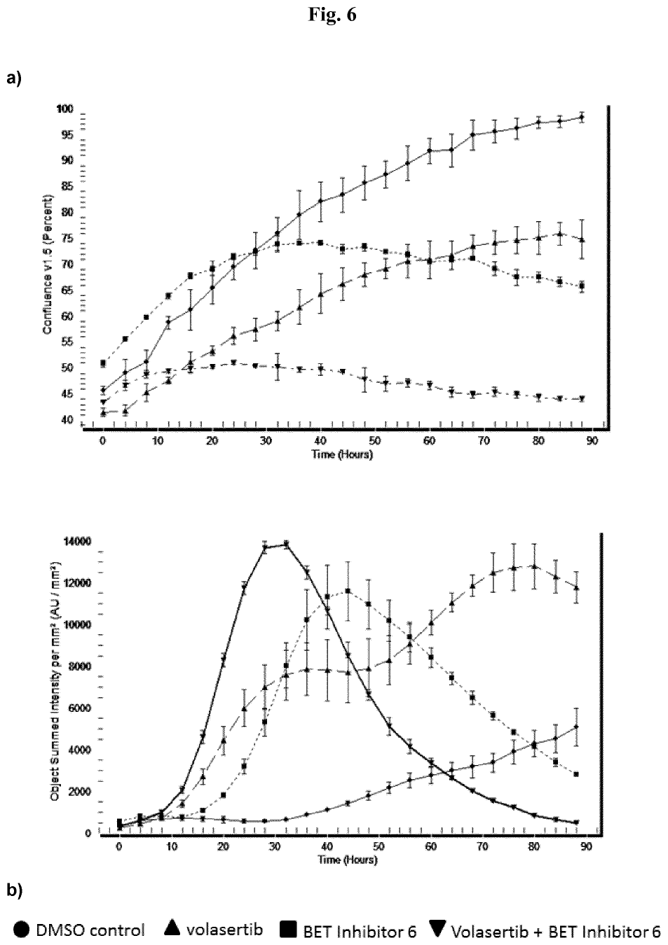

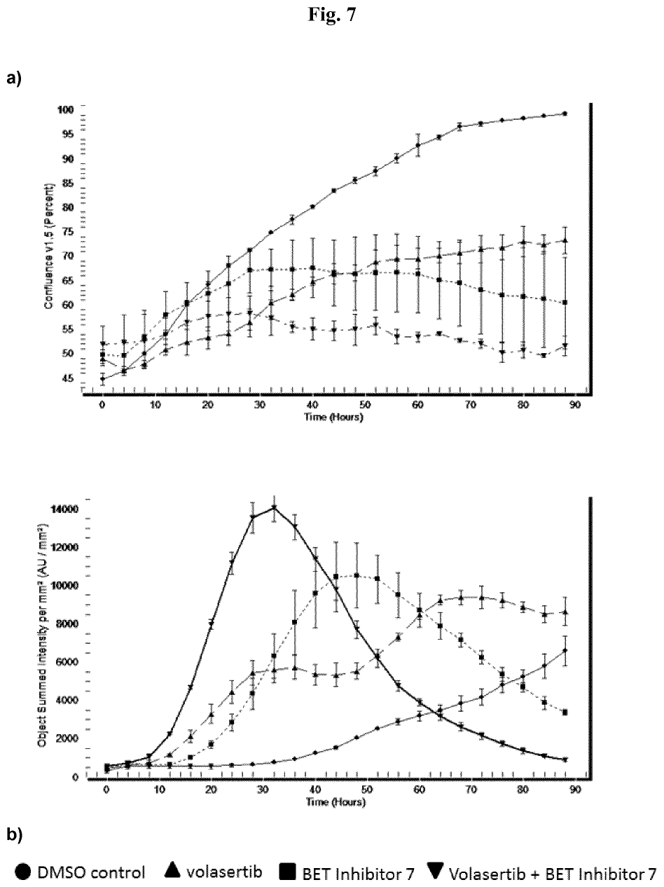

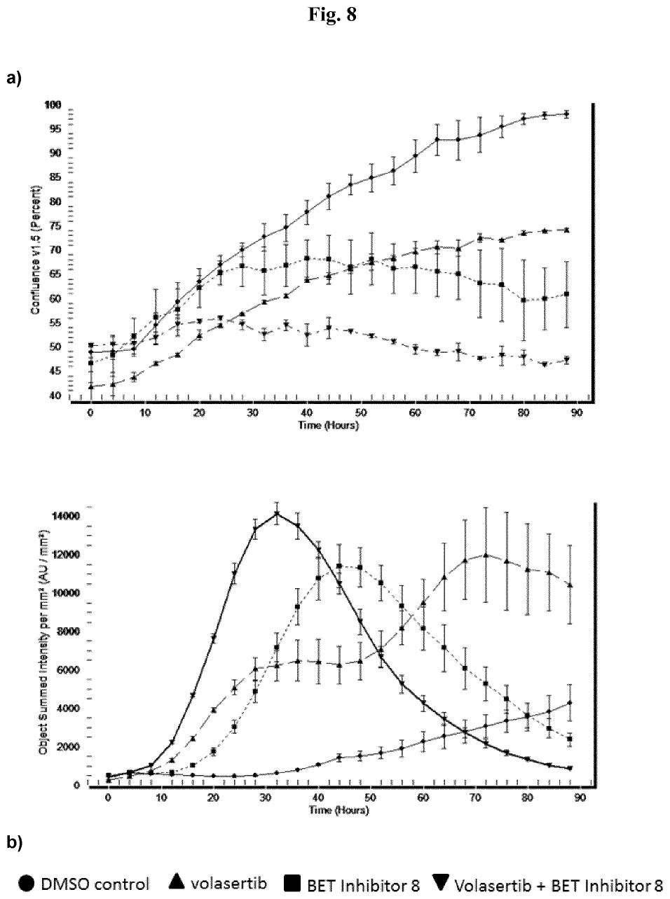

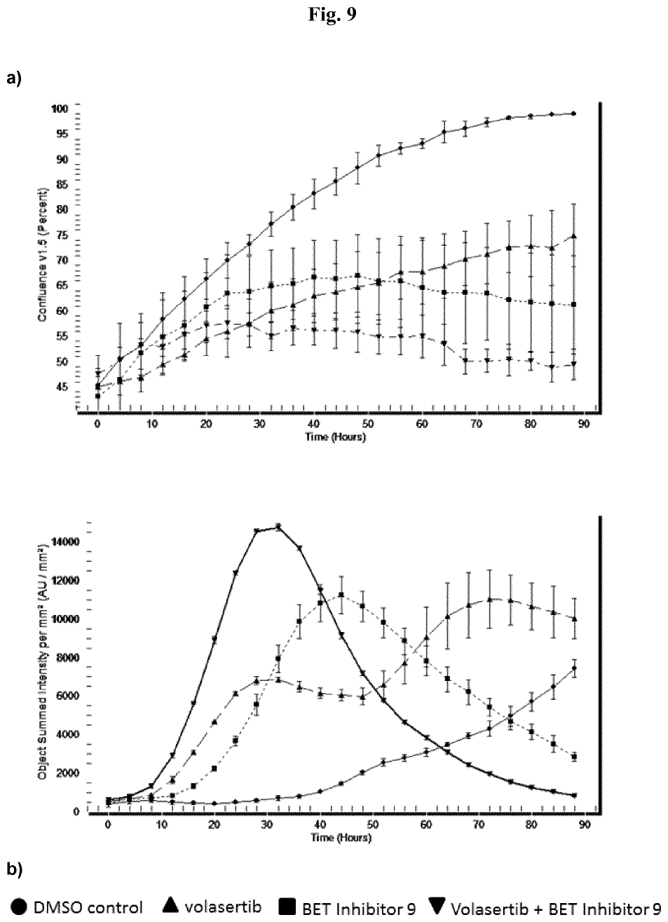

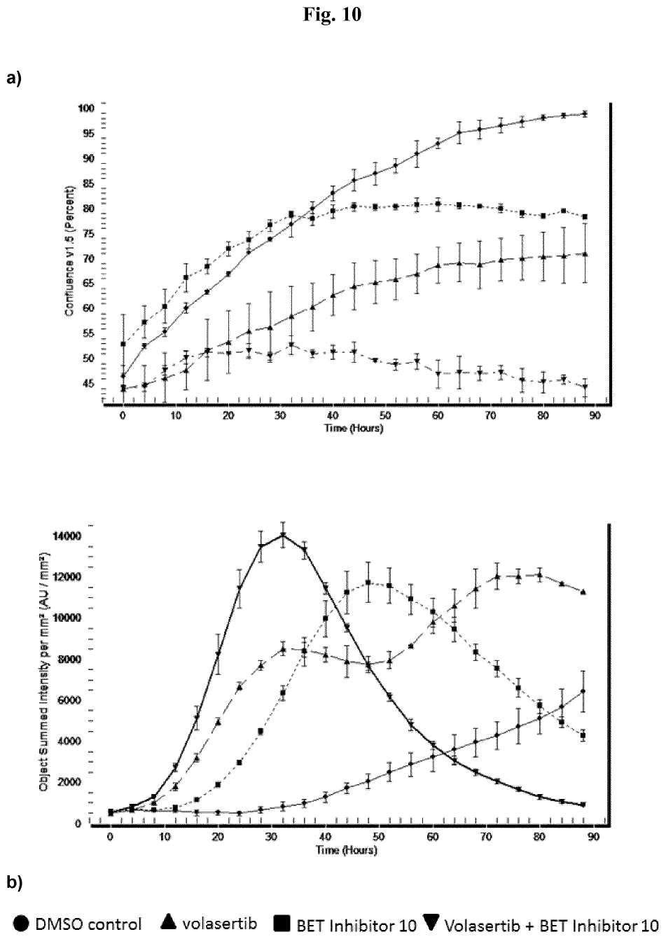

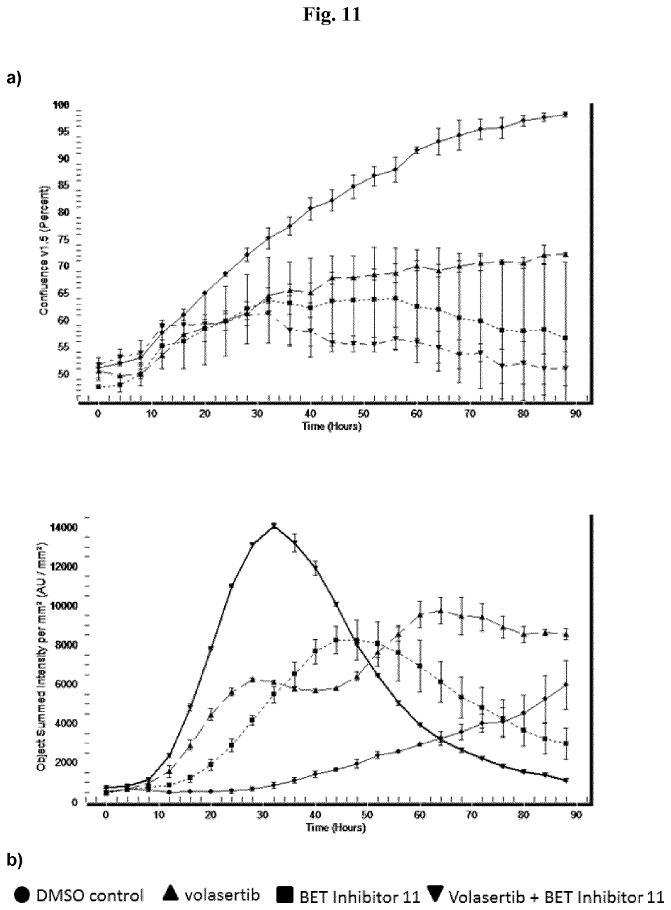

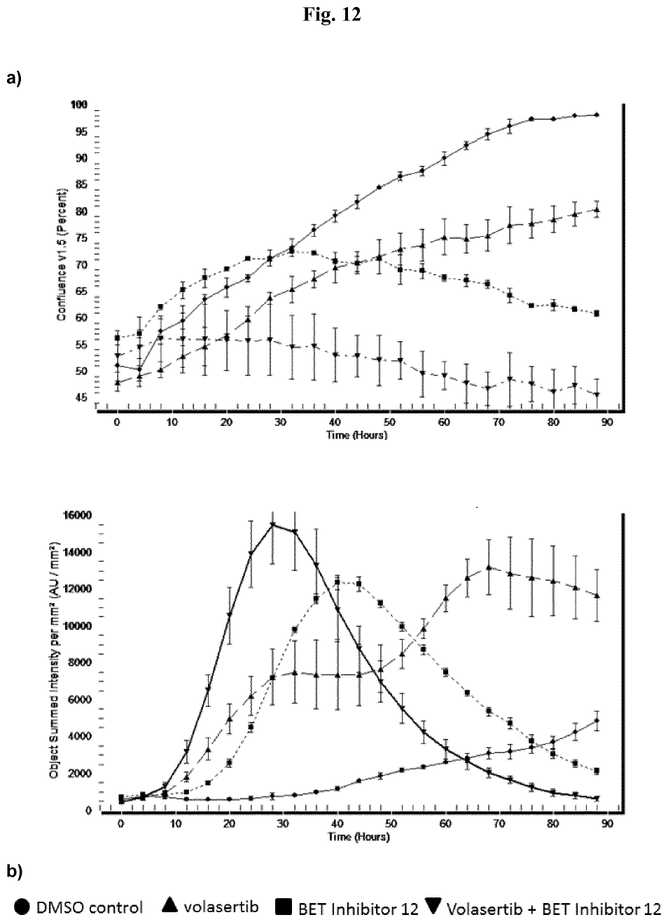

Each figure shows analysis of cell growth (a) and apoptosis (b) in AML cell line MV-4-11B over time for one of the exemplified BET inhibitors 1-12. The analysis is done by the Essen BioScience IncuCyte™ FLR live cell imaging system. It enables observation and quantification of cell behavior over time by automatically gathering and analyzing images around the clock. This live-cell, non-perturbating imaging approach yields kinetic data, all generated within the controlled environment of a standard cell incubator.

Cell growth (a) of BET inhibitor treated cells is reduced in comparison to DMSO control treated cells. Cell growth of volasertib treated cells is reduced in comparison to DMSO control treated cells. Combination of BET inhibitor plus volasertib treatment reduces cell proliferation more than each single treatment.

Apoptosis (b) of BET inhibitor treated cells is increased in comparison to DMSO control treated cells. Apoptosis of volasertib treated cells is increased in comparison to DMSO control treated cells. Combination of BET inhibitor plus volasertib treatment increased apoptosis more than each single treatment.

Cells

MV-4-11B is the AML cell line MV-4-11 from ATCC (CRL-9591) which has achieved a mutation in TP53 (c.742C>T, p.R248W, heterozygous for TP53). MV-4-11B cells were grown in T-75 flasks using RPMI1640 medium supplemented with 10% fetal calf serum and 50 μM mercaptoethanol. Cultures were incubated at 37° C. and 5% CO₂ in a humidified atmosphere.

Assay

For the IncuCyte live cell imaging assays, cells were plated in 96 well plates, Poly-D-Lysine coated, and were incubated with the respective compounds (BET inhibitor 1-12, volasertib), either alone or in combination. For the detection of apoptotic cells, the Essen BioSciences CellPlayer™ 96 well Kinetic Caspase-3/7 Reagent was added.

To determine cell proliferation, “confluence” (area covered with cells in the wells) was used as readout, to determine the amount of apoptosis (dead cells), the intensity of green fluorescence was used as readout.

### DETAILED DESCRIPTION OF THE INVENTION

Within the present invention the term “AML” is to be understood to encompass all forms of acute myeloid leukemia and related neoplasms according to the 2008 revision of the World Health Organization (WHO) classification of myeloid neoplasms and acute leukemia. These are:
Acute myeloid leukemia with recurrent genetic abnormalities
   AML with t(8;21)(q22;q22); RUNX1-RUNX1T1
   AML with inv(16)(p13.1q22) or t(16;16)(p13.1;q22); CBFB-MYH11
   AML with t(9;11)(p22;q23); MLLT3-MLL
   AML with t(6;9)(p23;q34); DEK-NUP214
   AML with inv(3)(q21q26.2) or t(3;3)(q21;q26.2); RPN1-EVI1
   AML (megakaryoblastic) with t(1;22)(p13;q13); RBM15-MKL1
   Provisional entity: AML with mutated NPM1
   Provisional entity: AML with mutated CEBPA
   AML with mutations: FLT3 ITD, KIT, N-RAS, MLL, WT1, IDH1/2, TET2, DNMT3A, ASXL1
Acute myeloid leukemia with myelodysplasia-related changes
Therapy-related myeloid neoplasms
Acute myeloid leukemia, not otherwise specified
   AML with minimal differentiation
   AML without maturation
   AML with maturation
   Acute myelomonocytic leukemia
   Acute monoblastic/monocytic leukemia
   Acute erythroid leukemia
      Pure erythroid leukemia
      Erythroleukemia, erythroid/myeloid
   Acute megakaryoblastic leukemia
   Acute basophilic leukemia
   Acute panmyelosis with myelofibrosis
Myeloid sarcoma
Myeloid proliferations related to Down syndrome
   Transient abnormal myelopoiesis
   Myeloid leukemia associated with Down syndrome
Blastic plasmacytoid dendritic cell neoplasm

In accordance with the present invention volasertib may be administered by parenteral (e.g. intramuscular, intraperitoneal, intravenous, transdermal or subcutaneous injection), and may be formulated, alone or together, in suitable dosage unit formulations containing conventional non-toxic pharmaceutically acceptable carriers, adjuvants and vehicles appropriate for each route of administration. Dosage forms and formulations of both active ingredients suitable within the present invention are known in the art. For instance, such dosage forms and formulations include those disclosed for volasertib in WO 2006/018221.

In accordance with the present invention the BET inhibitor may be administered by oral routes of administration and may be formulated, alone or together, in suitable dosage unit formulations containing conventional non-toxic pharmaceutically acceptable carriers, adjuvants and vehicles appropriate for each route of administration.

The following Examples serve to illustrate the invention without restricting it:

Synthesis of BET Inhibitors

The synthesis of BET inhibitors 5, 7, 11 and 13-51 is disclosed in patent application WO 2014/076237.

BET inhibitor 8 is known in the art and is disclosed in e.g. WO 2014/068402.

BET inhibitor 10 is known in the art and is disclosed in e.g. Journal of Medicinal Chemistry (2013), 56(19), 7501-7515.

BET inhibitors 52 and 53 are known in the art.

BET inhibitors 1-4, 6, 9 and 12 are synthetized as herein described.

### List of Abbreviations

| | |
|---|---|
| ACN, CH₃CN | acetonitrile |
| BINAP | 2,2′-bis(diphenylphosphino)-1,1′-binaphthyl |
| Boc | tert-butoxy carbonyl; di-tert-butyl dicarbonate |
| Boc₂O | Boc anhydride |
| CO | carbon monoxide |
| DCM | dichloromethane |
| dppf | 1,1′-bis(diphenylphosphino)ferrocene |
| DIPEA | diisopropylethyl amine |
| DMAP | dimethyl-pyridin-4-yl-amine |
| DMF | N,N-dimethylformamide |
| DMSO | dimethylsulphoxide |
| EDTA | ethylenediaminetetraacetic acid |
| EtOAc or EA | ethyl acetate |
| EtOH | ethanol |
| FCS | fetal calf serum |
| h | hour(s) |
| Hal | halogen |
| HATU | N-[(dimethylamino)-(1H-1,2,3-triazolo[4,5-b]pyridin-1- |
| | yl)-methylene]-N-methylmethan-aminium |
| | hexafluorophosphate N-oxide |
| HPLC | high performance liquid chromatography |
| K₂CO₃ | potassium carbonate |
| KOAc | potassium acetate |
| LiHMDS | lithium hexamethyl disilazide |
| M | molar (mol/L) |
| Min | minute(s) |
| ml | milliliter |
| MS | mass spectrometry |
| N | normal |
| Na₂SO₄ | sodium sulfate |
| NBS | N-bromo succinimide |
| NCS | N-chloro succinimide |
| NMR | nuclear resonance spectroscopy |
| Pd₂dba₃ | tris(dibenzylideneacetone)dipalladium(0) |
| Pd(dppf)Cl₂•CH₂Cl₂ | [1,1′- |
| | bis(diphenylphosphino)ferrocene]dichloropalladium(II), |
| | dichloromethane |
| PE | petrol ether |
| PPh₃ | triphenylphosphine |
| DIBAL | diisobutylaluminium hydride |
| RP | reversed phase |
| Rpm | rounds per minute |
| RT or rt | room temperature |
| SOCl₂ | thionyl chloride |
| STAB | sodium triacetoxy borohydride |
| TBACl | tetrabutylammonium chloride |
| TBME | tert-butyl methyl ether |
| TBTU | o-benzotriazol-1-yl-N,N,N′,N′-tetramethyluronium |
| | tetrafluoroborate |
| TEA | triethylamine |
| tert | tertiary |
| TFA | trifluoroacetic acid |
| THF | tetrahydrofuran |
| tR | retention time [min] |
| TRIS | tris(hydroxymethyl)aminomethane |
| wt % | weight percent |
| sat. | saturated |
| Ar | aromatic |

### GENERAL

Unless stated otherwise, all the reactions are carried out in commercially obtainable apparatus using methods that are commonly used in chemical laboratories. Starting materials that are sensitive to air and/or moisture are stored under protective gas and corresponding reactions and manipulations therewith are carried out under protective gas (nitrogen or argon).

The compounds are named according to the Beilstein rules using the Autonom software (Beilstein). If a compound is to be represented both by a structural formula and by its nomenclature, in the event of a conflict the structural formula is decisive.

Chromatography

Thin layer chromatography is carried out on ready-made TLC plates of silica gel 60 on glass (with fluorescence indicator F-254) made by Merck.

The preparative high pressure chromatography (HPLC) of the example compounds according to the invention is carried out with columns made by Waters (names: Sunfire C18 OBD, 10 μm, 30×100 mm Part. No. 186003971; X-Bridge C18 OBD, 10 μm, 30×100 mm Part. No. 186003930). The compounds are eluted using different gradients of H₂O/ACN wherein 0.2% HCOOH is added to the water (acid conditions). For chromatography under basic conditions the water is made basic according to the following recipe: 5 mL of ammonium hydrogen carbonate solution (158 g to 1 L H₂O) and 2 mL 32% ammonia (aq.) are made up to 1 L with H₂O.

The analytical HPLC (reaction monitoring) of intermediate compounds is carried out with columns made by Waters and Phenomenex. The analytical equipment is also provided with a mass detector in each case.

HPLC Mass Spectroscopy/UV Spectrometry

The retention times/MS-ESI⁺ for characterising the example compounds according to the invention are produced using an HPLC-MS apparatus (high performance liquid chromatography with mass detector) made by Agilent. Compounds that elute at the injection peak are given the retention time t_{Ret.}=0.

HPLC preparative methods

prep. HPLC1

| HPLC: | 333 and 334 Pumps | |
|---|---|---|
| Column: | Waters X-Bridge C18 OBD, 10 μm, 30 × 100 mm, Part. No. 186003930 | |
| Solvent: | A: 10 mM NH₄HCO₃ in H₂O; B: acetonitrile (HPLC grade) | |
| Detection: | UV/Vis-155 | |
| Flow: | 50 mL/min | |
| Gradient: | 0.00-1.50 min: | 1.5% B |
| | 1.50-7.50 min: | varying |
| | 7.50-9.00 min: | 100% B |
| prep. HPLC2 | | |
| HPLC: | 333 and 334 Pumps | |
| Column: | Waters Sunfire C18 OBD, 10 μm, 30 × 100 mm, Part. No. 186003971 | |
| Solvent: | A: H₂O + 0.2% HCOOH; B: acetonitrile (HPLC grade) + 0.2% HCOOH | |
| Detection: | UV/Vis-155 | |
| Flow: | 50 mL/min | |
| Gradient: | 0.00-1.50 min: | 1.5% B |
| | 1.50-7.50 min: | varying |
| | 7.50-9.00 min: | 100% B |

HPLC analytical methods

LCMS BAS1

| HPLC: | Agilent 1100 Series | |
|---|---|---|
| MS: | Agilent LC/MSD SL | |
| Column: | Phenomenex Mercury Gemini C18, 3 μm, 2 × 20 mm, | |
| | Part. No. 00M-4439-B0-CE | |
| Solvent: | A: 5 mM NH₄HCO₃/20 mM NH₃ in H₂O; B: acetonitrile (HPLC grade) | |
| Detection: | MS: Positive and negative mode | |
| Mass range: | 120-900 m/z | |
| Flow: | 1.00 mL/min | |
| Column temperature: | 40° C. | |
| Gradient: | 0.00-2.50 min: | 5% → 95% B |
| | 2.50-2.80 min: | 95% B |
| | 2.81-3.10 min: | 95% → 5% B |
| VAB | | |
| HPLC: | Agilent 1100/1200 Series | |
| MS: | Agilent LC/MSD SL | |
| Column: | Waters X-Bridge BEH C18, 2.5 μm, 2.1 × 30 mm XP | |
| Solvent: | A: 5 mM NH₄HCO₃/19 mM NH₃ in H₂O; B: acetonitrile (HPLC grade) | |
| Detection: | MS: Positive and negative mode | |
| Mass range: | 100-1200 m/z | |
| Flow: | 1.40 mL/min | |
| Column temperature: | 45° C. | |
| Gradient: | 0.00-1.00 min: | 5% → 100% B |
| | 1.00-1.37 min: | 100% B |
| | 1.37-1.40 min: | 100% → 5% B |
| METHOD 85_GVK | | |
| HPLC: | Water UPLC | |
| MS: | Micromass Triple quad | |
| Column: | Waters X-Bridge C18, 3.5 μm, 4.6 × 150 mm | |
| Solvent: | A: 10 mM NH₄HCO₃ in H₂O; B: acetonitrile (HPLC grade) | |
| Detection: | ES/APCI MODE | |
| Mass range: | 120-900 m/z | |
| Flow: | 1.00 mL/min | |
| Column temperature: | 25° C. | |
| Gradient: | 0.00-1.50 min: | 5% B |
| | 1.50-3.00 min: | 5% → 15% B |
| | 3.00-7.00 min: | 15% → 55% B |
| | 7.00-10.00 min: | 55% → 95% B |
| | 10.00-14.00 min: | 95% B |
| | 14.00-17.00 min: | 95% → 5% B |
| RND-FA-4.5-MIN | | |
| HPLC: | Water UPLC | |
| MS: | Micromass Triple quad | |
| Column: | Aquity UPLC BEH C18, 1.7 μm, 2.1 × 100 mm | |
| Solvent: | A: 0.1% formic acid in water, B: 0.1% formic acid in acetonitrile | |
| Detection: | ES/APCI MODE | |
| Flow: | 0.6 mL/min | |
| Column temperature: | 35° C. | |
| Gradient: | 0.00-0.40 min: | 3% B |
| | 0.40-3.20 min: | 3% → 98% B |
| | 3.20-3.80 min: | 98% B |
| | 3.80-4.20 min: | 98% → 3% B |
| | 4.20-4.50 min: | 3% B |

Synthesis of BET Inhibitors 1-3 and 12

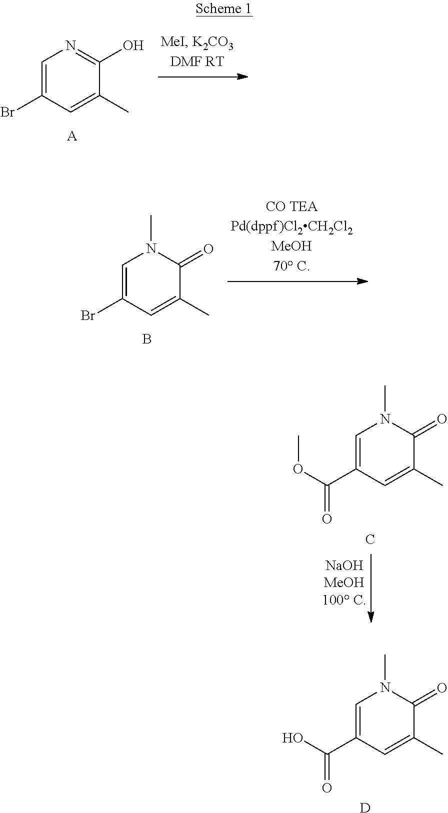

Synthesis of Intermediate B

To a suspension of starting material A (1.00 g; 5.053 mmol) and potassium carbonate (1.397 g; 10.105 mmol) in DMF (5 mL) is carefully added iodomethane (0.346 mL; 5.558 mmol). The reaction mixture is stirred overnight (16 h) at room temperature. The reaction mixture is then quenched with 10% ammonia solution (10 mL) and 30 mL water is added. It is extracted with 3×50 mL EtOAc. The combined organic layer is dried with Na₂SO₄, filtered and concentrated under reduced pressure to afford the product.

Yield: 98% (1.0 g; 4.95 mmol)

HPLC-MS: (M+H)⁺=202/204; t_{Ret}=0.65 min; method LCMS BAS1

Synthesis of Intermediate C

In the carbonylation reactor from BÜCHI GLAS USTER, intermediate B (3.30 g; 16.006 mmol) is dissolved in MeOH (80.00 mL) and TEA (5.399 mL; 40.015 mmol) is added. Then Pd(dppf)Cl₂.CH₂Cl₂ (389.00 mg; 0.476 mmol) is added and the reactor is closed and filled with carbon monoxide (8 bar). The reactor is heated to 70° C. and stirred overnight for 18 h. The reaction mixture is filtered through a small pad of silica and washed with ethyl acetate. The filtrate is concentrated under reduced pressure and the residue is purified on silica chromatography Combiflash (Column: Redisep Rf, 120 g; gradient: cHex/EtOAc=100%/0% to 50%/50%; flow rate=30 mL/min, 28 column volumes; detection wavelength: 254 nm). The product containing fractions are combined and concentrated under reduce pressure.

Yield: 90% (2.6 g; 14.35 mmol)

HPLC-MS: (M+H)⁺=182; t_{Ret}=0.49 min; method LCMS BAS1

Synthesis of Intermediate D

Intermediate C (2.60 g; 14.35 mmol) is suspended in MeOH. Sodium hydroxide (1 M solution, 45 mL; 45.00 mmol) is added and the reaction mixture is heated up to 100° C. (Drysyn, reflux) for 2 h. MeOH is removed under reduced pressure and 1 N HCl (46 mL) is added to the solution, precipitation occurs. The precipitate is filtered off and dried under reduced pressure.

Yield: 98% (2.34 g; 14.00 mmol)

HPLC-MS: (M+H)⁺=168; t_{Ret}=0 min; method LCMS BAS1

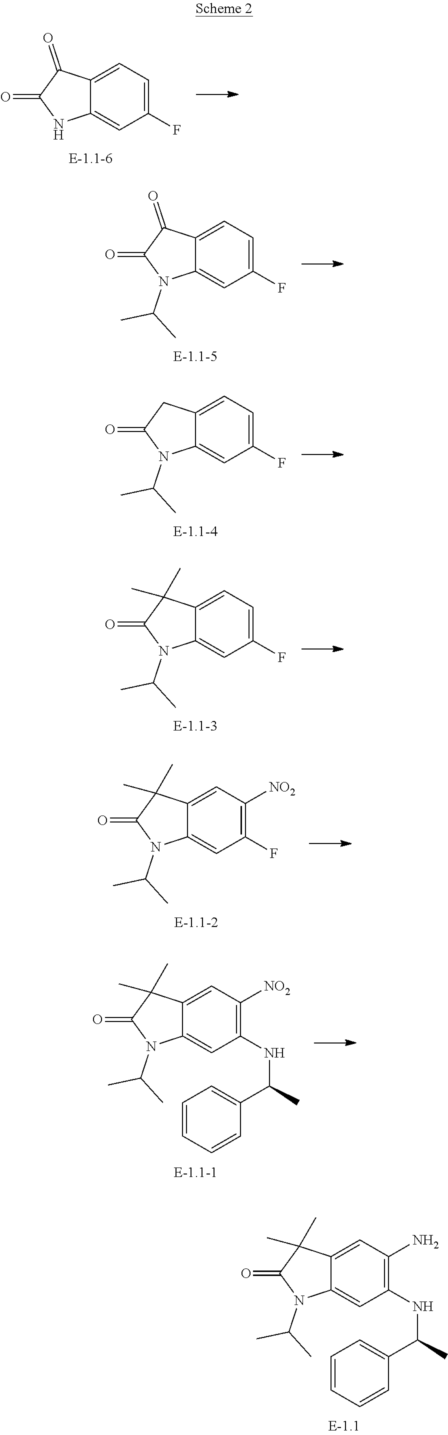

Synthesis of Intermediate E-1.1-5

To a stirred solution of starting material E-1.1-6 (55.0 g; 333.1 mmol) in DMF (550 mL) is added K₂CO₃ (55.2 g; 399.7 mmol) and 2-iodo-propane (73.6 g; 433.0 mmol) at 25° C. The reaction mixture is stirred at 25° C. for 16 h. Then the mixture is diluted with water and extracted with ethyl acetate. The organic layer is concentrated under reduced pressure to obtain crude compound. The crude is purified by column chromatography using silicagel (230-400 μm) with eluting mixture of ethyl acetate and hexane.

Yield: 51% (35.0 g; 169.0 mmol)

HPLC-MS: (M+H)⁺=208; t_{Ret}=2.02 min; method RND-FA-4.5-MIN

Synthesis of Intermediate E-1.1-4

To a stirred solution of intermediate E-1.1-5 (35.0 g; 169.0 mmol) in ethanol (1100 mL) hydrazine hydrate (550 mL) is added at 25° C. The reaction mixture is heated at 110° C. for 16 h. The reaction mixture is concentrated and the crude material is diluted with water and extracted with ethyl acetate. The organic layer is concentrated under reduced pressure to obtain the crude compound, which is purified by column chromatography using silicagel (230-400 μm) with eluting mixture of ethyl acetate and hexane.

Yield: 47% (15.3 g; 79.4 mmol)

HPLC-MS: (M+H)⁺=194; t_{Ret}=2.06 min; method RND-FA-4.5-MIN

Synthesis of Intermediate E-1.1-3

To a stirred solution of intermediate E-1.1-4 (24.0 g; 124.4 mmol) in THF (480 mL) is added sodium hydride (9.5 g; 397.5 mmol) portion wise at 0° C. The reaction mixture is stirred 25° C. for 20 min and then methyl iodide (24.8 mL; 385.1 mmol) is added drop wise. After 1 h stirring at 25° C. the reaction mixture is poured in to sat.NH₄Cl solution and extracted twice with ethyl acetate. The combined organic layers are dried over sodium sulfate and the solvent is evaporated under reduced pressure. The crude compound is purified by column chromatography using silica gel (230-400 μm) with eluting mixture of ethyl acetate and hexane.

Yield: 62% (17.0 g; 76.9 mmol)

HPLC-MS: (M+H)⁺=222; t_{Ret}=2.42 min; method RND-FA-4.5-MIN

Synthesis of Intermediate E-1.1-2

To a stirred solution of intermediate E-1.1-3 (17.0 g; 76.9 mmol) in acetic acid (550 mL) is added fuming HNO₃ (8.5 g; 134.9 mmol) at 25° C. The reaction mixture is stirred for 15 min at 25° C. and then concentrated H₂SO₄ (17.0 g; 173.5 mmol) is added and the mixture is stirred for 30 min at 25° C. After completion of the reaction the reaction mixture was poured in to ice cold water. The obtained solid was filtered and dried. The solid was recrystallized with ethyl acetate to obtain pure compound.

Yield: 49% (10.0 g; 37.6 mmol)

HPLC-MS: (M+H)+=267; t_{Ret}=10.77 min; method: METHOD 85_GVK

Synthesis of Intermediate E-1.1-1

Intermediate E-1.1-2 (4.5 g; 16.5 mmol) is dissolved in NMP (20 mL), DIPEA (4.0 mL; 22.3 mmol) and (S)-1-phenyl-ethylamine (2.65 mL; 20.1 mmol) is added at 20° C. The reaction mixture is stirred for 3 h at 50° C. The reaction mixture is poured into water and extracted with DCM. The combined organic layer is dried over MgSO₄ and concentrated under reduced pressure. The crude compound is purified by column chromatography using silica gel (50 μm) with eluting mixture of ethyl acetate and cyclohexane.

Yield 95% (5.76 g; 15.7 mmol)

HPLC-MS: (M+H)⁺=327; t_{Ret}=1.22 min; method LCMS BAS1

Synthesis of Intermediate E-1.1

Intermediate E-1.1-1 (5.76 g; 15.7 mmol) is dissolved in THF (50 mL) and filled into a BÜCHE autoclave. Raney-Ni (500 mg) is added and hydrogenated at 6 bar for 16 h. The reaction mixture is filtered through a plug of celite and the filtrate is concentrated under reduced pressure.

Yield 74% (3.770 g; 11.326 mmol)

HPLC-MS: (M+H)⁺=338; t_{Ret}=0.84 min; method VAB

Intermediate E-1.2 can be synthesized in analogy to the procedure of E-1.1.

| | | MS (M + H)⁺; | HPLC- |
|---|---|---|---|
| # | structure | t_{Ret.} HPLC [min] | method |
| E-1.2 | 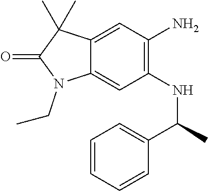 [SMILES: CCN1C(=O)C(C)(C)c2cc(N)c(N[C@@H](C)c3ccccc3)cc21] | M + H = 324; t_{Ret.} = 1.02 | VAB |

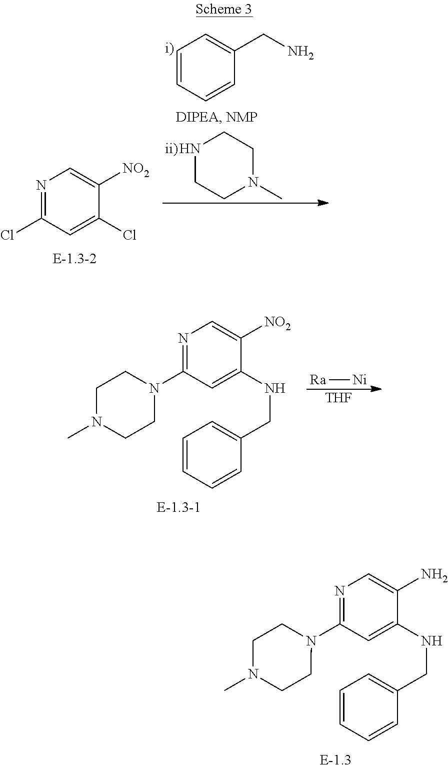

Synthesis of Intermediate E-1.3-1

To a solution of 2,4-dichloro-5-nitro-pyridine E-1.3-2 (5.00 g; 25.908 mmol) in NMP is added DIPEA (8.372 mL, 51.817 mmol) and benzylamine (3.054 mL, 28.499 mmol). The mixture is stirred for 1 h at RT. 1-Methylpiperazine (3.172 mL; 28.499 mmol) is then added and the mixture is stirred at 50° C. overnight. The residue is loaded onto isolute, split into five portions and purified using the basic preparatory reversed phase chromatography (method: prep. HPLC1). Product containing fractions are combined and freeze-dried (yield: 66%, 5.619 g; 17.163 mmol)

Synthesis of Intermediate E-1.3

Intermediate E-1.3-1 (400 mg; 1.222 mmol) is dissolved in THF (50 mL) and filled into a BÜCHE autoclave. Raney-Ni is added and the reaction is hydrogenated with 5 bar hydrogen pressure overnight. The reaction mixture is filtered on a plug of celite. The filtrate is then concentrated under reduced pressure. The product is used in the next step without further purification.

Yield: 74% (270 mg; 0.908 mmol)

HPLC-MS: (M+H)⁺=298; t_{Ret}=0.68 min; method VAB

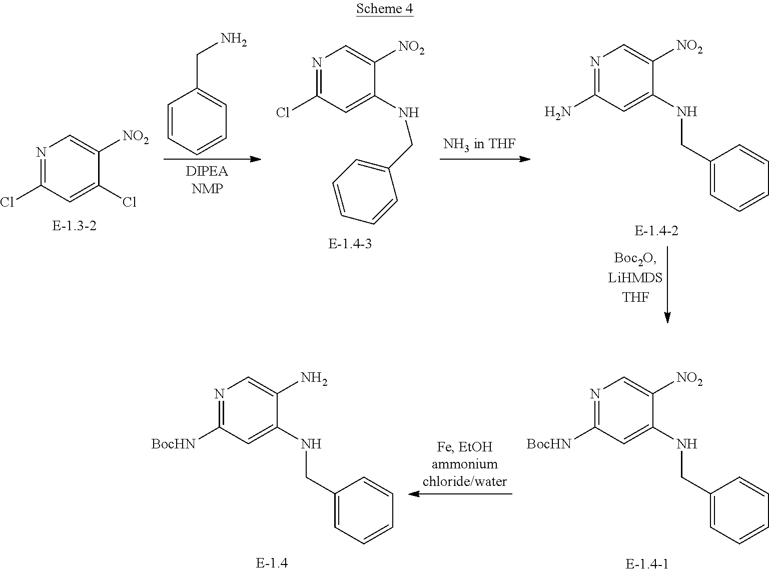

Synthesis of Intermediate E-1.4-3

To a stirred solution of E-1.3-2 (10.00 g; 51.817 mmol) and benzylamine (5.552 g; 51.817 mmol) in NMP is added DIPEA (20.053 g; 155.451 mmol) at 0° C. The mixture is stirred at RT for 1 h. Water is added, precipitation of the product occurs. Product is filtered off and dried under vacuum. The product is used in the next step without further purification (yield: 88%, 12.00 g; 45.510 mmol)

Synthesis of Intermediate E-1.4-2

A solution of intermediate E-1.4-3 (10.00 g; 38.02 mmol) in THF is placed in a steel bomb vessel. Liquid ammonia is added at −78° C. and the mixture is stirred at 90° C. for 16 h. The reaction is concentrated under reduced pressure. Water is added, precipitation of the product occurs. Product is filtered off and dried under vacuum. The residue is used in the next step without further purification (yield: 97%, 9.000 g; 36.85 mmol).

Synthesis of Intermediate E-1.4-1

LiHMDS (1M in THF, 55.271 mmol) is added at −78° C. to a solution of intermediate E-1.4-2 (9.00 g; 36.85 mmol) in THF. The mixture is stirred for 15 min at −78° C. Boc anhydride (8.836 g; 40.53 mmol) is then added and the mixture is stirred for 1 h at −78° C. The reaction mixture is quenched with aq. NH₄Cl solution, precipitation of the product occurs. Product is filtered off and dried under vacuum. The residue is used in the next step without further purification (yield: 55%, 7.00 g; 20.327 mmol).

Synthesis of Intermediate E-1.4

To a solution of intermediate E-1.4-1 (7.00 g; 20.327 mmol) in ethanol is added a solution of ammonium chloride (5.427 g, 102 mmol) in water and iron powder (5.671 g; 102 mmol). The reaction is stirred at 80° C. for 2 h. The reaction mixture is filtered through celite. The filtrate is concentrated under reduced pressure. The residue is purified by flash column chromatography on basic alumina using 1-2 MeoH/DCM as eluent. The isolated product is obtained as brown colour solid. It is taken for the next step without further purification.

Yield: 70% (4.50 g; 14.314 mmol)

TLC (10% MeOH/90% DCM): Rf=0.09

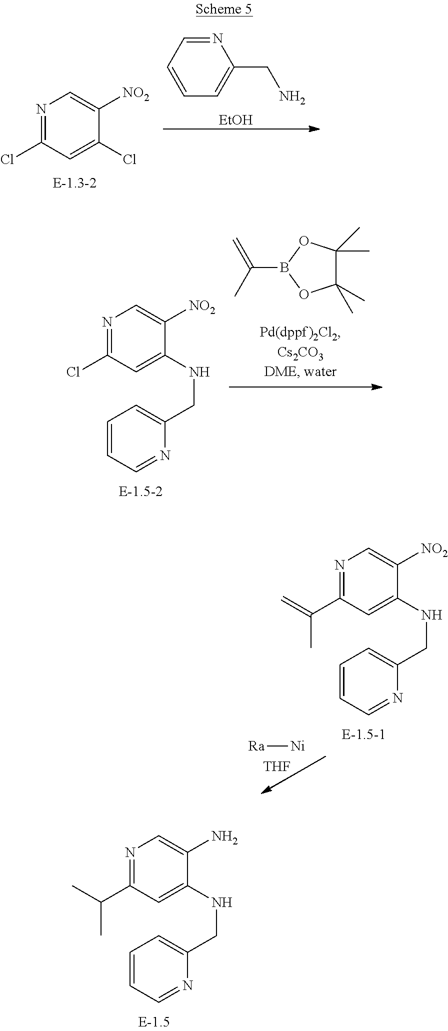

Synthesis of Intermediate E-1.5-2

E-1.5-2 is synthesized in analogy to the procedure described for the synthesis of E-1.4-3.

Synthesis of Intermediate E-1.5-1

Intermediate E-1.5-2 (125 mg; 0.47 mmol), 4,4,5,5-tetramethyl-2-(prop-1-en-2-yl)-1,3,2-dioxaborolane (200 μL; 1.06 mmol), cesium carbonate (300 mg; 0.90 mmol) and [1,1′-bis[diphenylphosphino]-ferrocene]dichloropalladium (30 mg; 0.04 mmol) are suspended in 7.5 mL dimethoxyethane and 2.5 mL water and heated for 1 h at 100° C. The solvent of the reaction mixture is removed under reduced pressure and the crude product is purified using reversed phase chromatography (Method: prep. HPLC1).

Yield: 43% (55 mg; 0.20 mmol)

HPLC-MS: (M+H)⁺=271; t_{Ret}=1.09 min; method LCMS BAS1

Synthesis of Intermediate E-1.5

E-1.5 is synthesized in analogy to the procedure described for the synthesis of E-1.3 from E-1.3-1.

The following intermediates are synthesized in analogy to the procedures of E-1.3, E-1.4 and E-1.5.

| | | MS (M + H)⁺; | HPLC- |
|---|---|---|---|
| # | structure | t_{Ret.} HPLC [min] | method |
| E-1.6 | 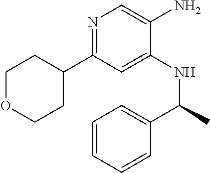 [SMILES: C[C@H](Nc1cc(C2CCOCC2)ncc1N)c1ccccc1] | M + H = 298; t_{Ret.} = 0.80 | VAB |
| E-1.7 | 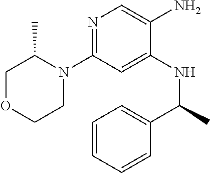 [SMILES: C[C@H](Nc1cc(N2CCOC[C@@H]2C)ncc1N)c1ccccc1] | M + H = 313; t_{Ret.} = 1.60 | FECB5 |

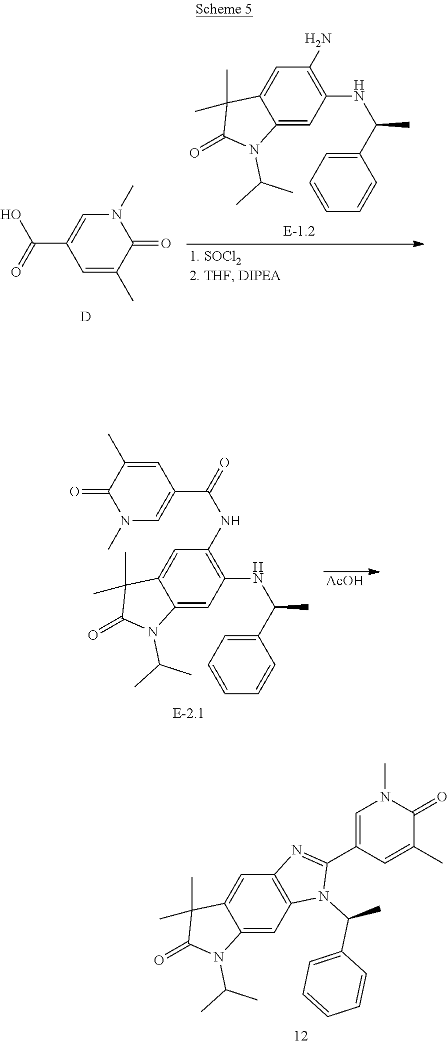

Synthesis of Intermediate E-2.1

Intermediate D (52 mg; 0.31 mmol) is suspended in 30 mL thionyl chloride and heated for 15 h at 60° C. The reaction mixture is concentrated under reduced pressure. Intermediate E-1.2 (105 mg; 0.31 mmol) is dissolved in 5 mL THF and DIPEA (162 μL; 1.00 mmol) is added. To this reaction mixture the acid chloride dissolved in 2 mL THF is added and the resulting reaction mixture is stirred for 12 h at 20° C. The solvent is evaporated and the crude product is purified using reversed phase chromatography (Method: prep. HPLC1).

Yield: 94% (140 mg; 0.29 mmol)

HPLC-MS: (M+H)⁺=487; t_{Ret}=1.22 min; method LCMS BAS1

Synthesis of BET inhibitor 12

Intermediate E-2.1 (100 mg; 0.21 mmol) is dissolved in 2 mL acetic acid and stirred at 120° C. for 7 h. The solvent is evaporated and the crude product is purified using reversed phase chromatography (Method: prep. HPLC2).

Yield: 38% (37 mg; 0.08 mmol)

HPLC-MS: (M+H)⁺=469; t_{Ret}=1.20 min; method LCMS BAS1

BET inhibitors 1 to 3 are synthesized in analogy to the procedure of BET inhibitor 12

| | | MS (M + H)⁺; | |
|---|---|---|---|
| # | structure | t_{Ret.} HPLC [min] | HPLC-method |
| 1 | 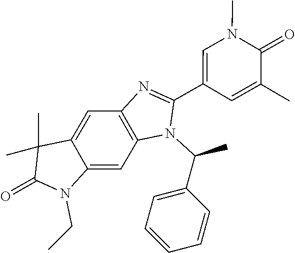 [SMILES: CCN1C(=O)C(C)(C)c2cc3nc(-c4cc(C)c(=O)n(C)c4)n([C@@H](C)c4ccccc4)c3cc21] | M + H = 455; t_{Ret.} = 1.13 | LCMS BAS1 |
| 2 | 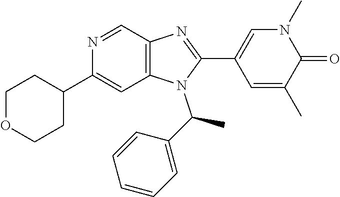 [SMILES: Cc1cc(-c2nc3cnc(C4CCOCC4)cc3n2[C@@H](C)c2ccccc2)cn(C)c1=O] | M + H = 429; t_{Ret.} = 1.02 | LCMS BAS1 |
| 3 | 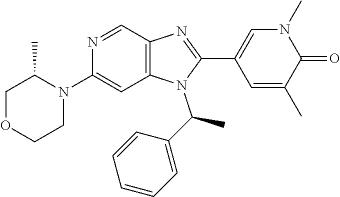 [SMILES: Cc1cc(-c2nc3cnc(N4CCOC[C@@H]4C)cc3n2[C@@H](C)c2ccccc2)cn(C)c1=O] | M + H = 444; t_{Ret.} = 1.08 | LCMS BAS1 |

Synthesis of BET Inhibitors 4, 6 and 9

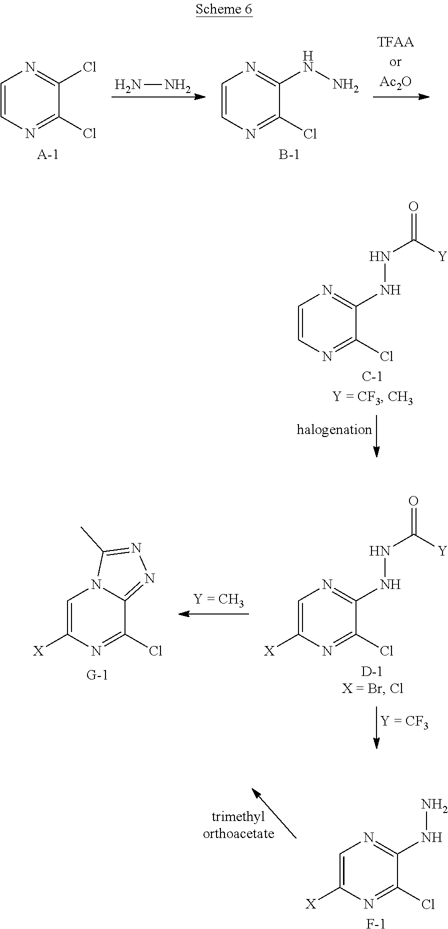

Synthesis of Intermediate B-1

Starting material A-1 (15 g; 100.68 mmol) and hydrazine hydrate 65% (15.509 mL; 201.37 mmol) are dissolved in 45 mL ethanol and stirred for 1 h at 80° C. While cooling down, a precipitate is formed. It is slurred up with a small amount of water and filtered off. It is washed with water and then dried to afford the product.

Yield: 93% (13.6 g; 94.07 mmol)

HPLC-MS: (M+H)⁺=145/147; t_{Ret}=0.34 min; method FECB5

Synthesis of Intermediate C-1a

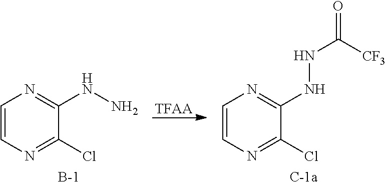

Intermediate B-1 (15.6 g; 108 mmol) is slurried up in THF (300 mL) and cooled down in an ice bath to −5° C. Trifluoroacetic anhydride (17 mL; 118 mmol) is also dissolved in 300 mL THF and dropped slowly to the first solution. After 1 h most of the THF is evaporated, then a small amount of water is added and the mixture is extracted with DCM. The organic phase is dried over MgSO₄ and evaporated to dryness.

Yield: 100%

HPLC-MS: (M+H)⁺=241/243; t_{Ret}=1.31 min; method FSUN2

Synthesis of Intermediate C-1b

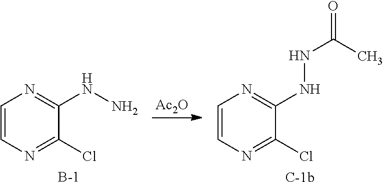

Intermediate B-1 (15.0 g; 104 mmol) is suspended in toluene (135 mL) at ambient temperature. Acetic anhydride (11.1 g; 109 mmol) dissolved in 7.5 mL toluene is slowly added to the suspension. The reaction mixture is stirred at ambient temperature for 2.5 h. The product is isolated by filtration.

Yield: 99% (19.2 g, 103 mmol)

ESI-MS: (M+H)⁺=187/189

Synthesis of Intermediate D-1a

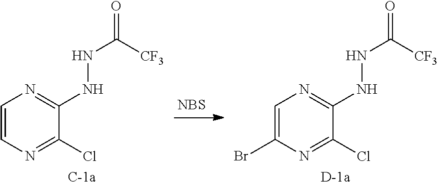

Intermediate C-1a (19.5 g; 81.1 mmol) is dissolved in 300 mL anhydrous DCM and cooled down to −40° C. Afterwards NBS (18.8 g; 105 mmol) is added and stirred for 1 h. The solution is diluted with water and extracted with DCM. The organic phase is then purified with flash chromatography: cHex/(EtOAc/CH₃COOH=9/1)=80%/20% to 70%/30% within 10 column volumes.

Yield: 11% (2.83 g; 8.859 mmol)

HPLC-MS: (M−H)⁻=317/319/321; t_{Ret}=1.79 min; method FSUN2

Synthesis of Intermediate D-1b

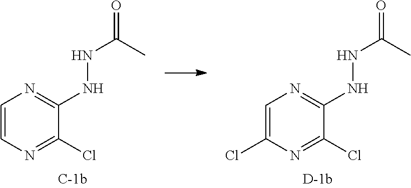

To a suspension of intermediate C-1b (4.0 g; 21.4 mmol) in 15 mL EtOAc is added 0.30 g (1.1 mol) TBACl at ambient temperature. After addition of a solution of NCS (2.9 g; 21.4 mmol) in 60 mL AcOEt the reaction mixture is stirred for 1 h. The solution is stirred for about 1.5 h while a precipitation is formed. The product is isolated by filtration.

Yield: 23% (1.1 g; 5.0 mmol)

ESI-MS: (M−H)⁺=221/223/225

Synthesis of Intermediate F-1a

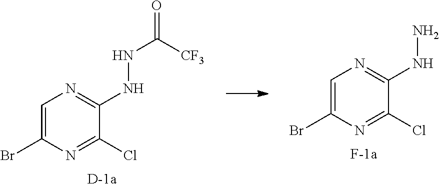 [SMILES: O=C(NNc1ncc(Br)nc1Cl)C(F)(F)F ; NNc1ncc(Br)nc1Cl]

Intermediate D-1a (1.59 g; 4.97 mmol) is dissolved in 30 mL EtOH and treated with 3 mL conc. HCl. It is stirred for 2 h at 100° C. The reaction mixture is cooled down, diluted with water and then the pH adjusted to 8 with saturated NaHCO₃ solution. The water phase is extracted with EtOAc, the organic layer dried over MgSO₄ and evaporated to dryness.

Yield: 71% (945 mg; 3.51 mmol)

HPLC-MS: (M−H)⁻=221/223/225; t_{Ret}=1.32 min; method FECB5

Synthesis of Intermediate G-1a

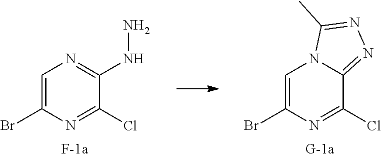

Intermediate F-1a (945 mg; 3.51 mmol) is dissolved in 12 mL trimethyl orthoacetate and heated up to 130° C. for 1 h. The solution is diluted with water and extracted with EtOAc. The organic phase is then purified with flash chromatography: cHex/EtOAc=70%/30% to 55%/45% within 10 column volumes.

Yield: 71% (824 mg; 3.33 mmol)

HPLC-MS: (M+H)⁺=247/249/251; t_{Ret}=1.23 min; method FECB5

Synthesis of Intermediate G-1b

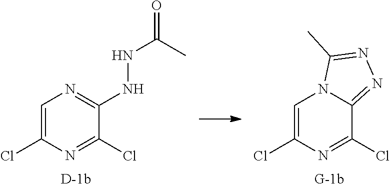

Intermediate D-1b (5.0 g; 22.6 mmol) is dissolved in 20 mL ACN and heated to reflux. Then, SOCl₂ (3.5 g, 29.4 mmol) is added. The reaction mixture is stirred for about 1 h at reflux temperature and 4 h at 0° C. The product is isolated by filtration.

Yield: 96% (4.4 g; 21.6 mmol)

ESI-MS: (M+H)⁺=203/205/207

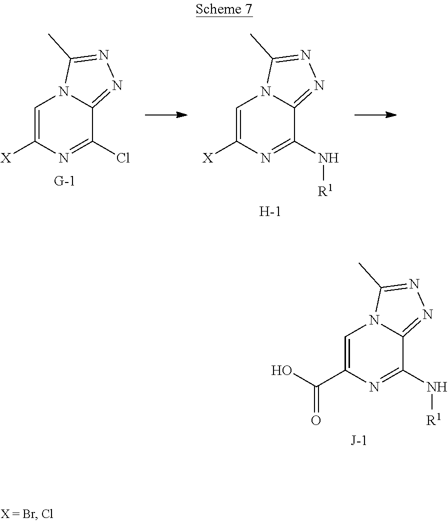

Synthesis of Intermediate H-1a

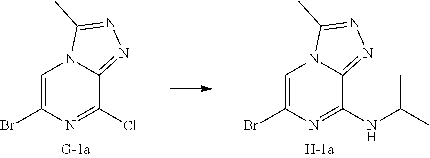

Intermediate G-1a (30.00 g; 121.2 mmol), isopropylamine (14.30 g; 242.44 mmol) and HÜNIG base (17.20 g; 133.33 mmol) are dissolved in 150 mL NMP and are stirred for 2 h at 80° C. The reaction mixture is diluted with water and extracted with EtOAc. The organic layer is separated and dried over MgSO₄ and evaporated to dryness.

Yield: 70% (42.00 g; 155.55 mmol)

HPLC-MS: (M+H)⁺=270/272; t_{Ret}=0.69 min; method VAB

Synthesis of Intermediate H-1 b

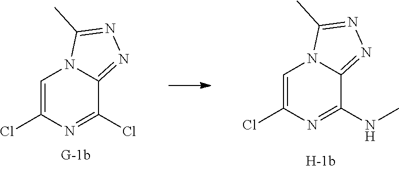

To a solution of G-1b (3.29 g; 16.2 mmol) in 10.0 mL NMP is added methylylamine 33% in EtOH (6.1 g; 65.4 mmol) and the reaction mixture is stirred for 1.5 h. The reaction mixture is diluted with 50 mL water. The product is isolated by filtration.

Yield: 88% (2.82 g; 14.3 mmol)

ESI-MS: (M+H)⁺=198/200

Synthesis of Intermediate H-1c

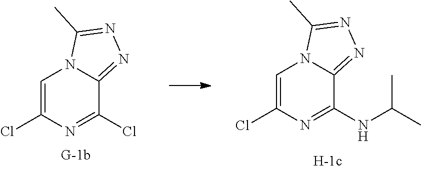

Intermediate G-1b (5.0 g; 24.6 mmol) is dissolved in 50 mL water and cooled to 0° C. Then, isopropylylamine (7.28 g; 123 mmol) is added and the mixture is stirred for 5 h at 0° C. The product is isolated by filtration.

Yield: 80% (4.5 g; 19.8 mmol)

ESI-MS: (M+H)⁺=226/228

Synthesis of Intermediate J-1a

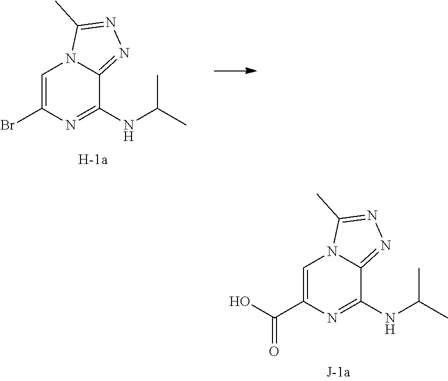 [SMILES: Cc1nnc2c(NC(C)C)nc(Br)cn12 ; Cc1nnc2c(NC(C)C)nc(C(=O)O)cn12]

Intermediate H-1a (10 g; 37.02 mmol), dichloro[1,1′-bis(diphenylphosphino)ferrocene] palladium (II) dichloro-methane adduct (3.02 g, 3.70 mmol) and triethylamine (9.35 g; 92.57 mmol) are dissolved in 50 mL methanol and 50 mL NMP. The reaction mixture is stirred for 2 h at 130° C. and 2 bar CO pressure. The reaction mixture is diluted with water and extracted with EtOAc. The organic layer is separated and dried over MgSO₄ and evaporated to dryness. The crude product is purified using method prep. HPLC1. The intermediate obtained is dissolved in 100 mL THF and is treated with 100 mL of a 1 N aqueous LiOH solution. After 1 h the reaction mixture is diluted with water and extracted with DCM. The organic layer is separated and dried over MgSO₄ and evaporated to dryness.

Yield: 57% (5 g; 21.27 mmol)

HPLC-MS: (M+H)⁺=236; t_{Ret}=0.0 min; method VAB

Synthesis of Intermediate E-1.8

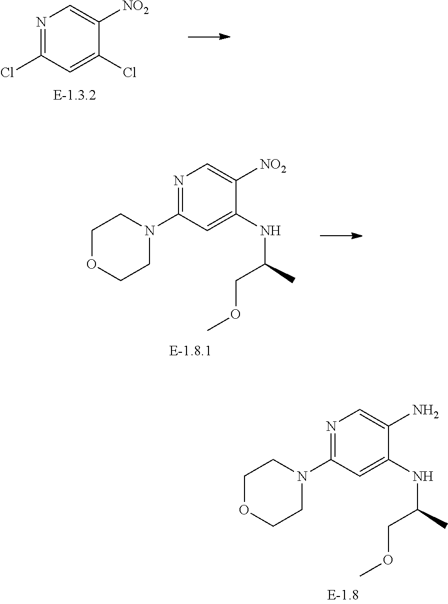 [SMILES: O=[N+]([O-])c1cnc(Cl)cc1Cl ; COC[C@H](C)Nc1cc(N2CCOCC2)ncc1[N+](=O)[O-] ; COC[C@H](C)Nc1cc(N2CCOCC2)ncc1N]

Starting material E-1.3.2 (500 mg; 2.46 mmol), (2S)-1-methoxypropan-2-amine (219 mg; 2.46 mmol) and triethylamine (400 μL; 2.82 mmol) are suspended in 2.5 mL NMP and stirred for 1 h at 25° C. To this suspension morpholine (500 μL; 5.71 mmol) is added and the resulting mixture is stirred for 16 h at 50° C. The crude intermediate is purified using reversed phase chromatography (prep. HPLC1). This intermediate is dissolved in 30 mL THF and palladium on carbon is added. The reaction mixture is stirred for 3 h at 25° C. and 4 bar hydrogen pressure. The solid material is filtered off and the solvent is evaporated.

Yield: 65% (422 mg; 1.59 mmol)

HPLC-MS: (M+H)⁺=267; t_{Ret}=0.59 min; method VAB

Synthesis of Intermediate E-1.9

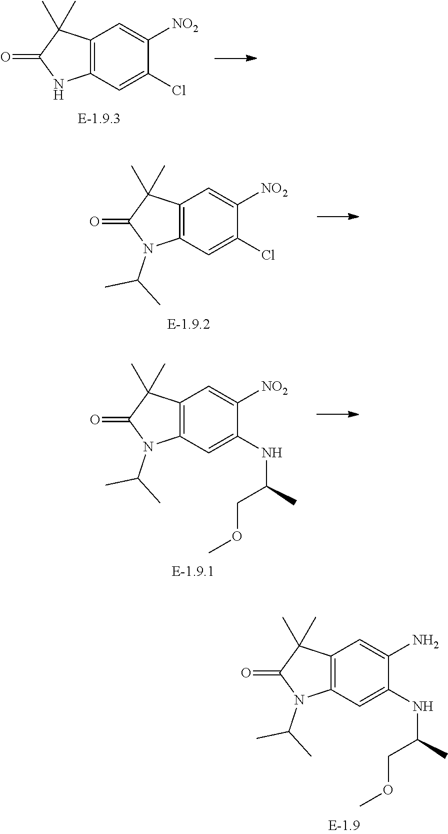 [SMILES: CC1(C)C(=O)Nc2cc(Cl)c([N+](=O)[O-])cc21 ; CC(C)N1C(=O)C(C)(C)c2cc([N+](=O)[O-])c(Cl)cc21 ; COC[C@H](C)Nc1cc2c(cc1[N+](=O)[O-])C(C)(C)C(=O)N2C(C)C ; COC[C@H](C)Nc1cc2c(cc1N)C(C)(C)C(=O)N2C(C)C]

To a solution of 6-chloro-3,3-dimethyl-5-nitro-1,3-dihydro-indol-2-one E-1.9.3 (250 mg; 1.18 mmol) in THF super dry (6 mL) sodiumhydride (188 mg; 4.70 mmol) is added at 0° C. Then 2-iodo-propane (696 mg; 4.12 mmol) is added at 0° C. and the reaction mixture is stirred at 25° C. for 18 h. The reaction mixture is poured on water and extracted twice with DCM. The organic phase is dried over MgSO₄ and concentrated under reduced pressure. The resulting residue is purified via normal phase chromatography (column: Interchim IR50-SI 40 g; Flow: 40 mL/min; mobile phase: cyclohexane/ethylacetate=1:1).

The intermediate obtained, E-1.9.2, (200 mg; 0.79 mmol) and (S)-2-methoxy-1-methyl-ethylamine (425 mg; 4.76 mmol) are dissolved in 1 mL NMP and stirred for 18 h at 80° C. The crude intermediate is purified using reversed phase chromatography (prep. HPLC1). The intermediate obtained, E-1.9.1, is dissolved in 30 mL THF and palladium on carbon is added. The reaction mixture is stirred for 3 h at 25° C. and 4 bar hydrogen pressure. The solid material is filtered off and the solvent is evaporated.

Yield: 42% (149 mg; 0.49 mmol)

HPLC-MS: (M+H)⁺=306; t_{Ret}=0.81 min; method VAB

Intermediate E-1.10 is synthesized in analogy to the procedure of intermediates E-1.8 and E-1.9

| | | MS (M + H)⁺; | HPLC- |
|---|---|---|---|
| # | structure | t_{Ret.} HPLC [min] | method |
| E-1.10 | 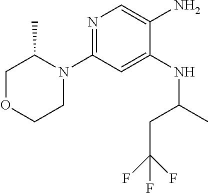 [SMILES: CC(CC(F)(F)F)Nc1cc(N2CCOC[C@@H]2C)ncc1N] | M + H = 319; t_{Ret.} = 0.78 | VAB |

Synthesis of BET inhibitor 4

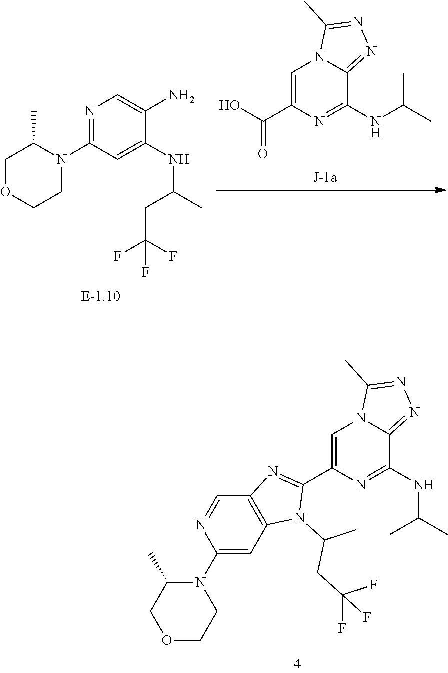 [SMILES: CC(CC(F)(F)F)Nc1cc(N2CCOC[C@@H]2C)ncc1N ; Cc1nnc2c(NC(C)C)nc(C(=O)O)cn12 ; Cc1nnc2c(NC(C)C)nc(-c3nc4cnc(N5CCOC[C@@H]5C)cc4n3C(C)CC(F)(F)F)cn12]

3-methyl-8-[(propan-2-yl)amino]-[1,2,4]triazolo[4,3-a]pyrazine-6-carboxylic acid J-1a (58 mg; 0.25 mmol), HÜNIG base (125 μL; 0.77 mmol) and HATU (100 mg; 0.26 mmol) are dissolved in 2 mL NMP. The reaction mixture is stirred for 10 min, then E-1.10 (63 mg; 0.25 mmol) is added and the resulting mixture is stirred for an additional 5 h at 25° C. The reaction mixture is diluted with water and DCM. The organic layer is separated and dried over MgSO₄ and the solvent is evaporated. The crude intermediate is dissolved in 4 mL acetic acid and stirred at 160° C. for 2 h. Afterwards the reaction mixture is neutralized with aqueous NaHCO₃ solution and extracted with DCM. The organic layer is separated and dried over MgSO₄ and the solvent is evaporated. The crude product is purified using reversed phase chromatography (Method: prep. HPLC1).

Yield: 25% (32 mg; 0.06 mmol)

HPLC-MS: (M+H)⁺=518; t_{Ret}=1.14 min; method LCMSBAS1 The following examples are synthesized in analogy to the procedure of BET inhibitor 4

| | | MS | |
|---|---|---|---|
| | | (M + H)⁺; | |
| | | t_{Ret.} HPLC | HPLC- |
| # | structure | [min] | method |
| 9 | 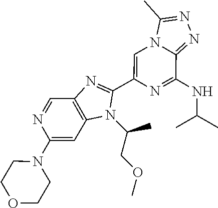 [SMILES: COC[C@H](C)n1c(-c2cn3c(C)nnc3c(NC(C)C)n2)nc2cnc(N3CCOCC3)cc21] | M + H = 466; t_{Ret.} = 1.02 | LCMSBAS1 |
| 6 | 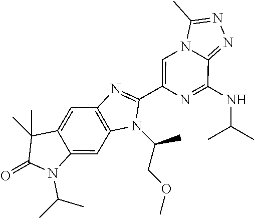 [SMILES: COC[C@H](C)n1c(-c2cn3c(C)nnc3c(NC(C)C)n2)nc2cc3c(cc21)N(C(C)C)C(=O)C3(C)C] | M + H = 505; t_{Ret.} = 1.22 | LCMSBAS1 |

1. A method of treating AML comprising administering to a patient in need of such treatment a therapeutically effective amount of volasertib, or a pharmaceutically acceptable salt thereof or a hydrate thereof, in combination with a therapeutically effective amount of a BET inhibitor, or a pharmaceutically acceptable salt thereof or a hydrate thereof, wherein both active ingredients are administered simultaneously, separately or sequentially.

2. (canceled)

3. The method according to claim 1, characterized in that volasertib is administered in combination with a BET inhibitor, or a pharmaceutically acceptable salt thereof or a hydrate thereof, according to a dosage schedule comprising:
a) administration of a therapeutically effective amount of volasertib, or a pharmaceutically acceptable salt thereof or a hydrate thereof, at least one day during a treatment cycle and
b) administration of a therapeutically effective amount of a BET inhibitor, or a pharmaceutically acceptable salt thereof or a hydrate thereof, on at least one day of said treatment cycle
to a patient suffering from AML.

4. The method according to claim 1, wherein the BET inhibitor is a diazepine derivative.

5. The method according to claim 1, wherein the BET inhibitor is a triazolopyrazine derivative.

6. The method according to claim 1, wherein the BET inhibitor is a pyridinone derivative.

7. The method according to claim 1, wherein the BET inhibitor is selected from:

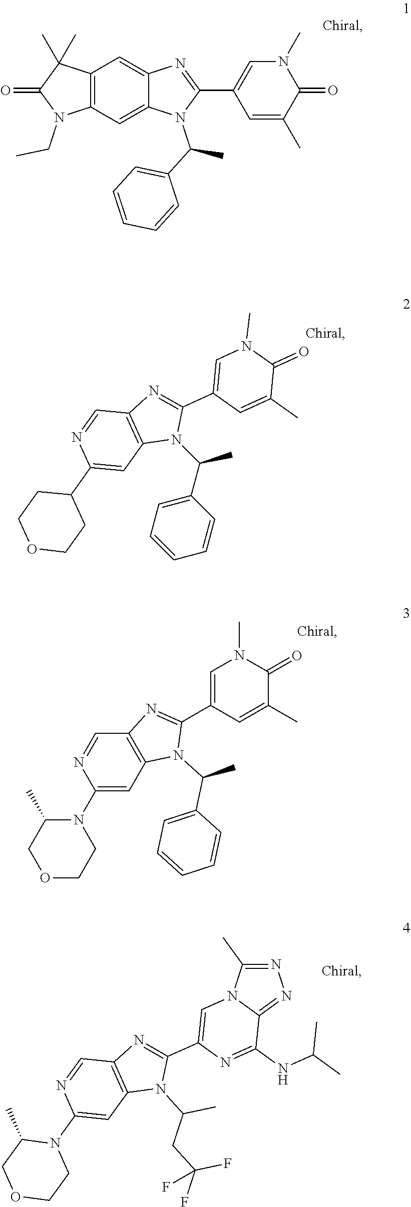 [SMILES: CCN1C(=O)C(C)(C)c2cc3nc(-c4cc(C)c(=O)n(C)c4)n([C@@H](C)c4ccccc4)c3cc21 ; Cc1cc(-c2nc3cnc(C4CCOCC4)cc3n2[C@@H](C)c2ccccc2)cn(C)c1=O ; Cc1cc(-c2nc3cnc(N4CCOC[C@@H]4C)cc3n2[C@@H](C)c2ccccc2)cn(C)c1=O ; Cc1nnc2c(NC(C)C)nc(-c3nc4cnc(N5CCOC[C@@H]5C)cc4n3C(C)CC(F)(F)F)cn12]

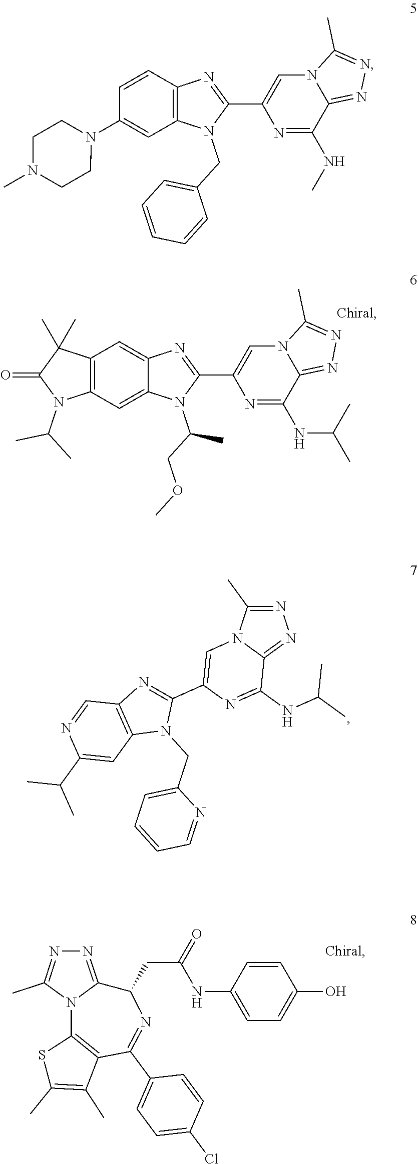

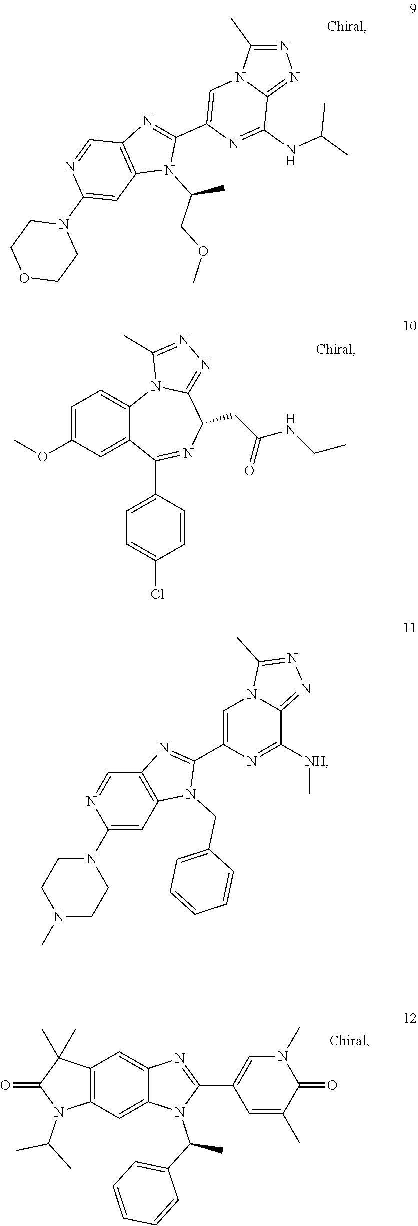

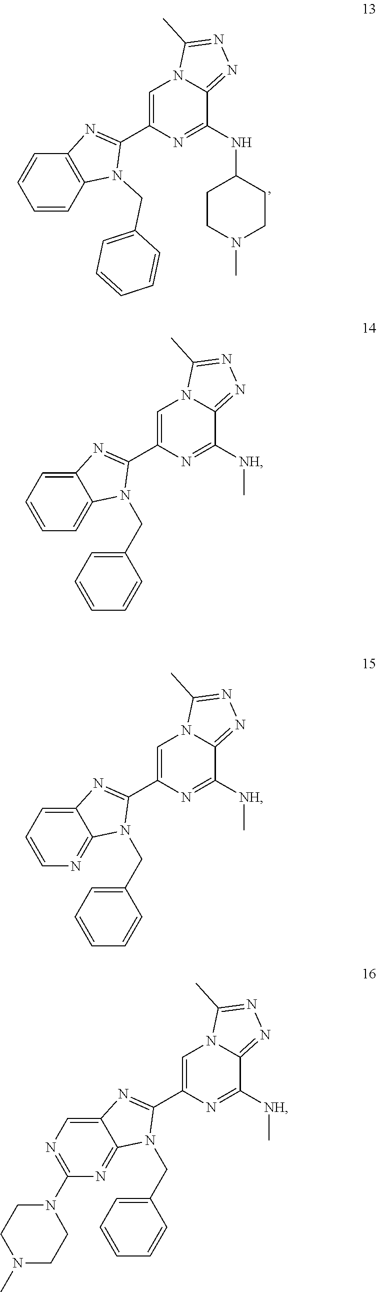 [SMILES: Cc1nnc2c(NC3CCN(C)CC3)nc(-c3nc4ccccc4n3Cc3ccccc3)cn12 ; CNc1nc(-c2nc3ccccc3n2Cc2ccccc2)cn2c(C)nnc12 ; CNc1nc(-c2nc3cccnc3n2Cc2ccccc2)cn2c(C)nnc12 ; CNc1nc(-c2nc3cnc(N4CCN(C)CC4)nc3n2Cc2ccccc2)cn2c(C)nnc12]

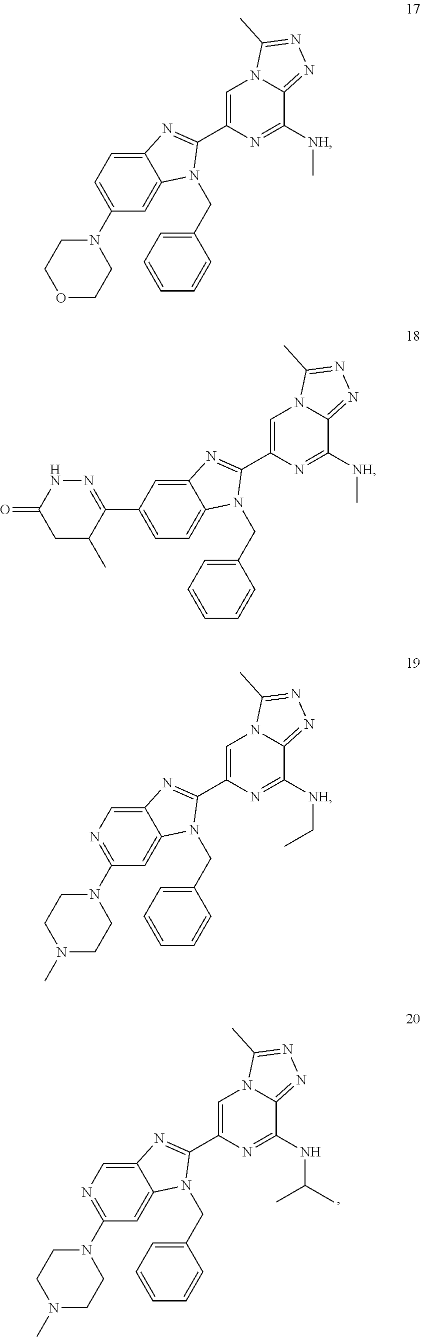 [SMILES: CNc1nc(-c2nc3ccc(N4CCOCC4)cc3n2Cc2ccccc2)cn2c(C)nnc12 ; CNc1nc(-c2nc3cc(C4=NNC(=O)CC4C)ccc3n2Cc2ccccc2)cn2c(C)nnc12 ; CCNc1nc(-c2nc3cnc(N4CCN(C)CC4)cc3n2Cc2ccccc2)cn2c(C)nnc12 ; Cc1nnc2c(NC(C)C)nc(-c3nc4cnc(N5CCN(C)CC5)cc4n3Cc3ccccc3)cn12]

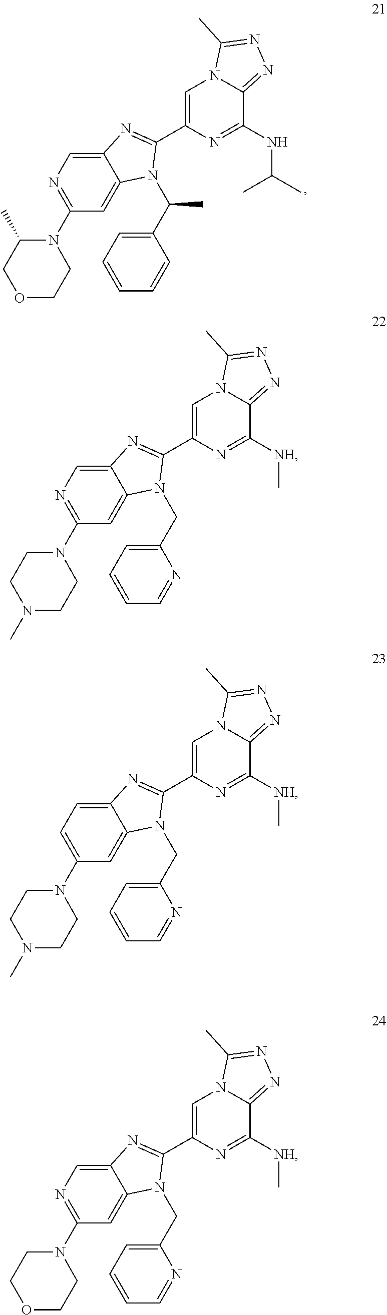 [SMILES: Cc1nnc2c(NC(C)C)nc(-c3nc4cnc(N5CCOC[C@@H]5C)cc4n3[C@@H](C)c3ccccc3)cn12 ; CNc1nc(-c2nc3cnc(N4CCN(C)CC4)cc3n2Cc2ccccn2)cn2c(C)nnc12 ; CNc1nc(-c2nc3ccc(N4CCN(C)CC4)cc3n2Cc2ccccn2)cn2c(C)nnc12 ; CNc1nc(-c2nc3cnc(N4CCOCC4)cc3n2Cc2ccccn2)cn2c(C)nnc12]

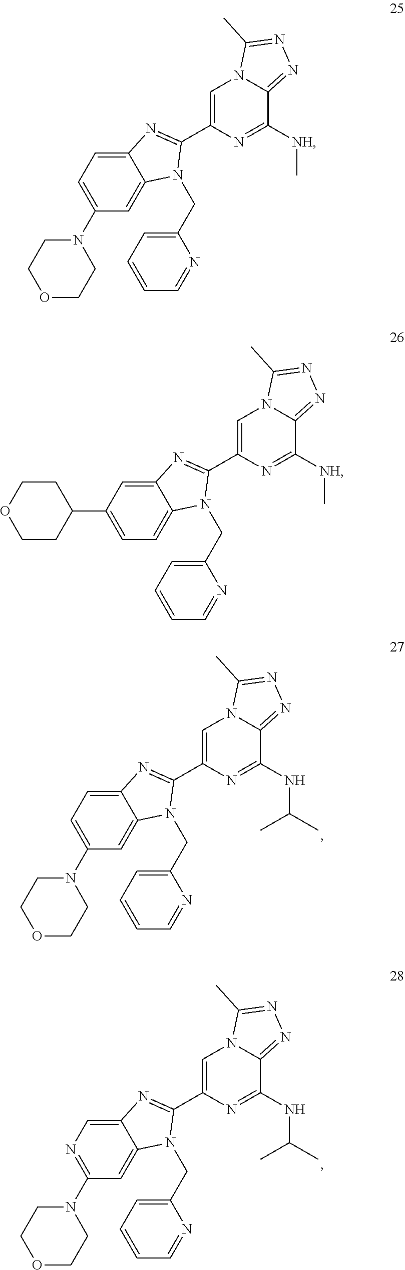

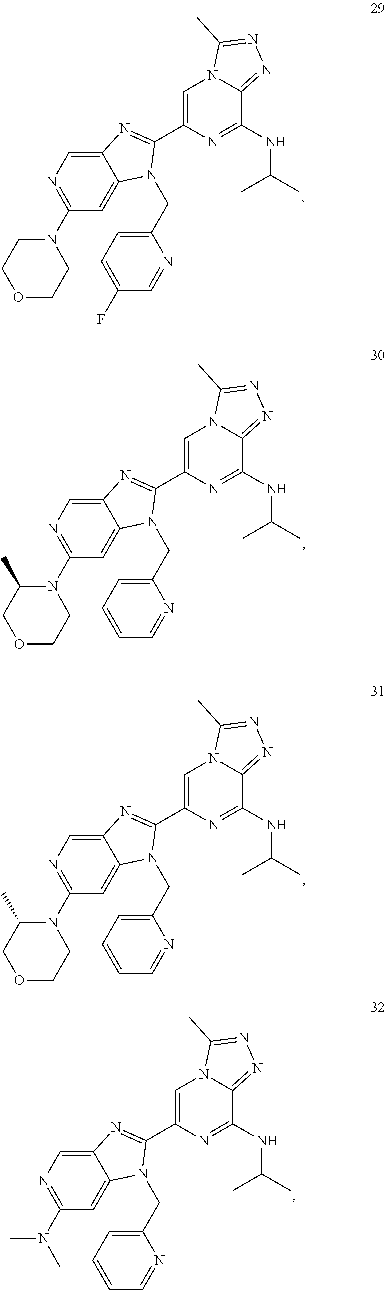

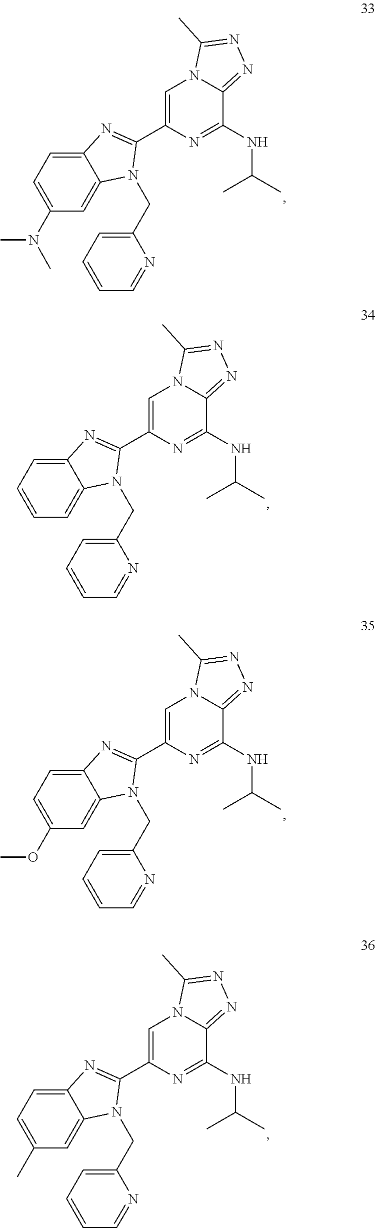

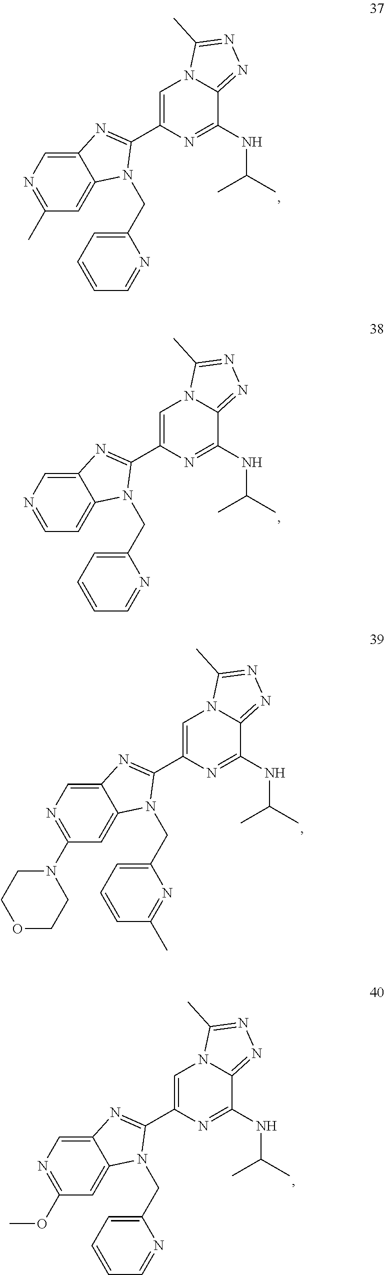

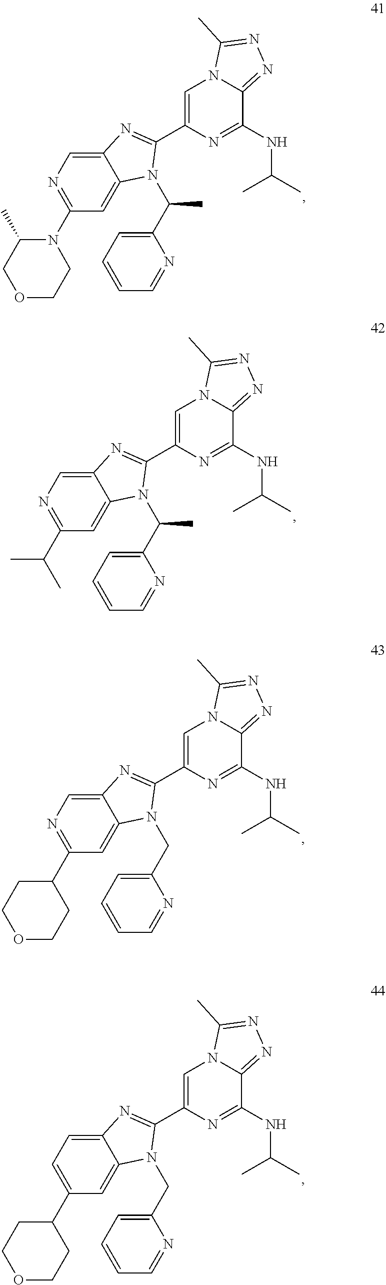

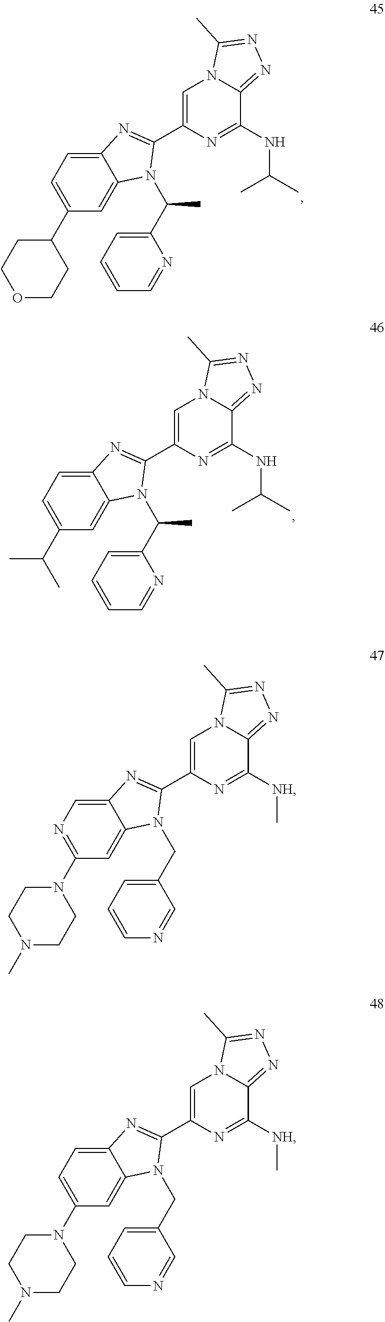

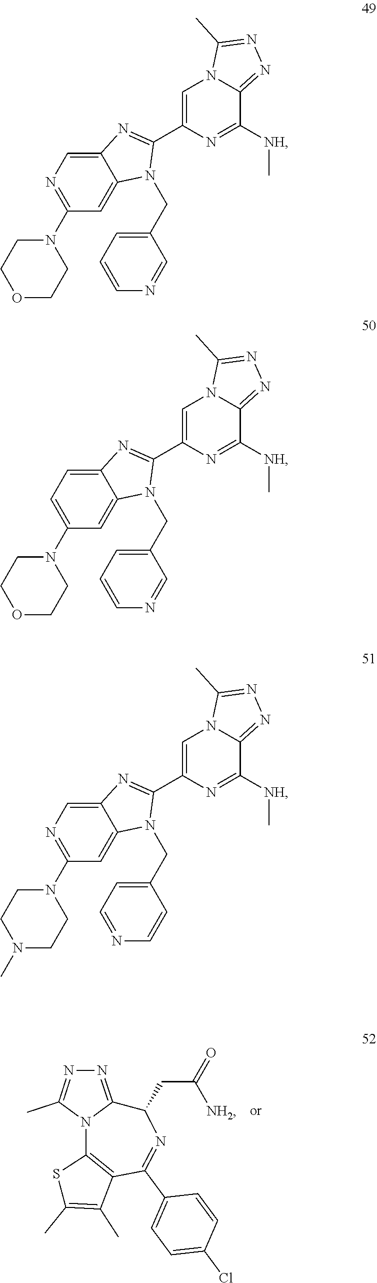

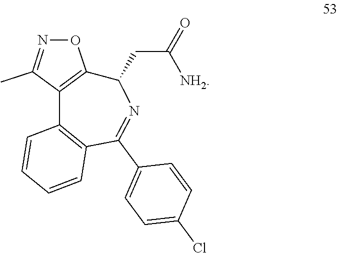 [SMILES: Cc1noc2c1-c1ccccc1C(c1ccc(Cl)cc1)=N[C@H]2CC(N)=O]

8. A pharmaceutical composition comprising a therapeutically effective amount of volasertib, or a pharmaceutically acceptable salt thereof or a hydrate thereof, and a therapeutically effective amount of a BET inhibitor, or a pharmaceutically acceptable salt thereof or a hydrate thereof.

9. The pharmaceutical composition according to claim 8, wherein the BET inhibitor, or a pharmaceutically acceptable salt thereof or a hydrate thereof, is selected from a diazepine derivative, a pyridinone derivative or a triazolopyrazine derivative.

10. The pharmaceutical composition according to claim 8, wherein the BET inhibitor, or a pharmaceutically acceptable salt thereof or a hydrate thereof, is selected from

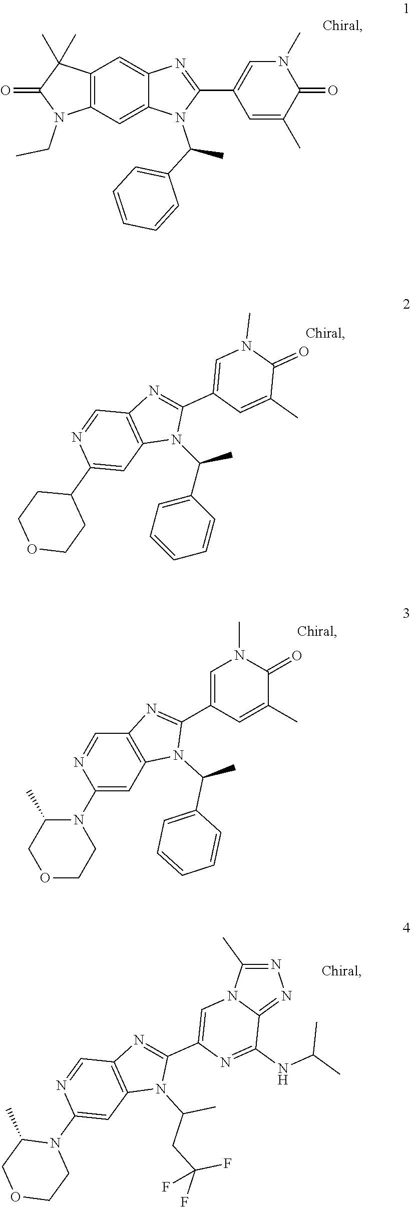 [SMILES: CCN1C(=O)C(C)(C)c2cc3nc(-c4cc(C)c(=O)n(C)c4)n([C@@H](C)c4ccccc4)c3cc21 ; Cc1cc(-c2nc3cnc(C4CCOCC4)cc3n2[C@@H](C)c2ccccc2)cn(C)c1=O ; Cc1cc(-c2nc3cnc(N4CCOC[C@@H]4C)cc3n2[C@@H](C)c2ccccc2)cn(C)c1=O ; Cc1nnc2c(NC(C)C)nc(-c3nc4cnc(N5CCOC[C@@H]5C)cc4n3C(C)CC(F)(F)F)cn12]

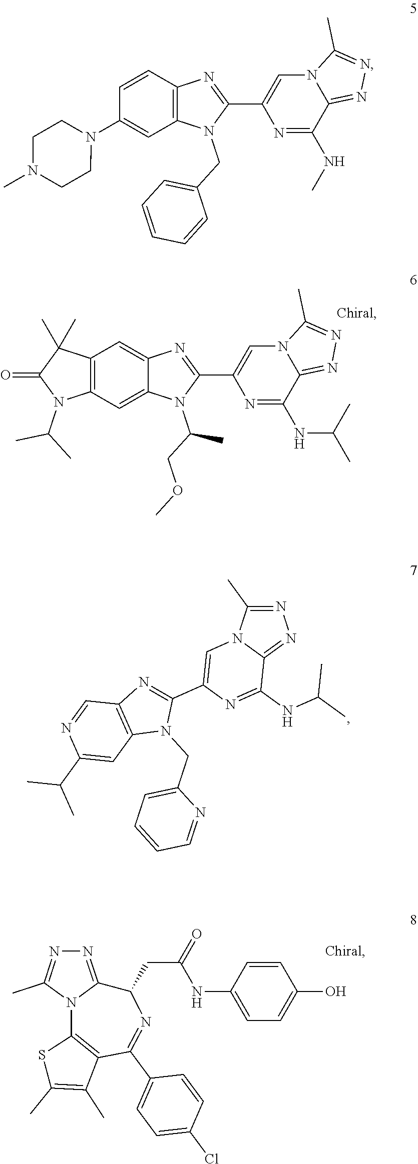

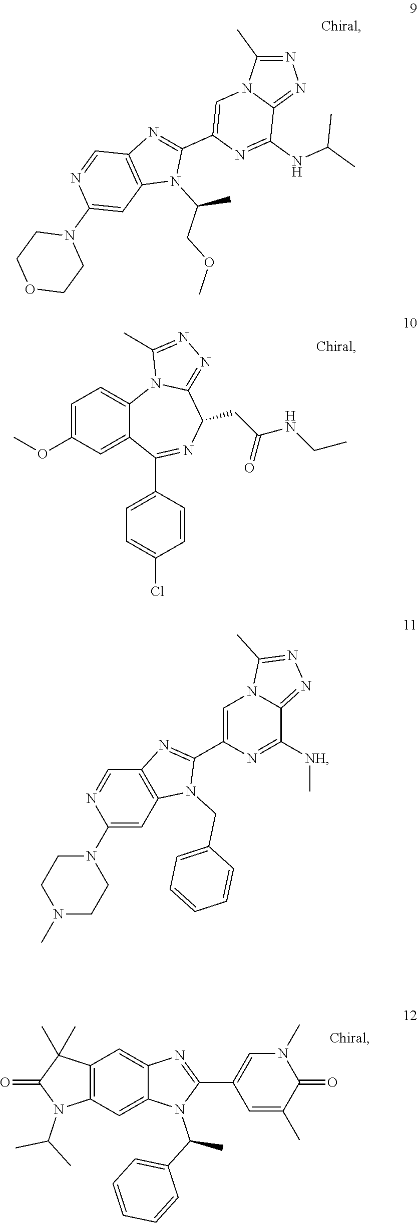

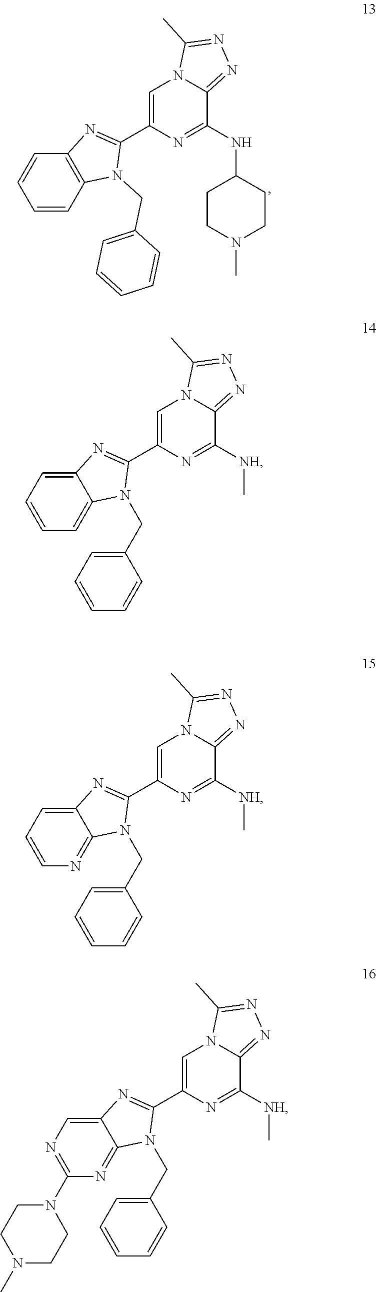 [SMILES: Cc1nnc2c(NC3CCN(C)CC3)nc(-c3nc4ccccc4n3Cc3ccccc3)cn12 ; CNc1nc(-c2nc3ccccc3n2Cc2ccccc2)cn2c(C)nnc12 ; CNc1nc(-c2nc3cccnc3n2Cc2ccccc2)cn2c(C)nnc12 ; CNc1nc(-c2nc3cnc(N4CCN(C)CC4)nc3n2Cc2ccccc2)cn2c(C)nnc12]

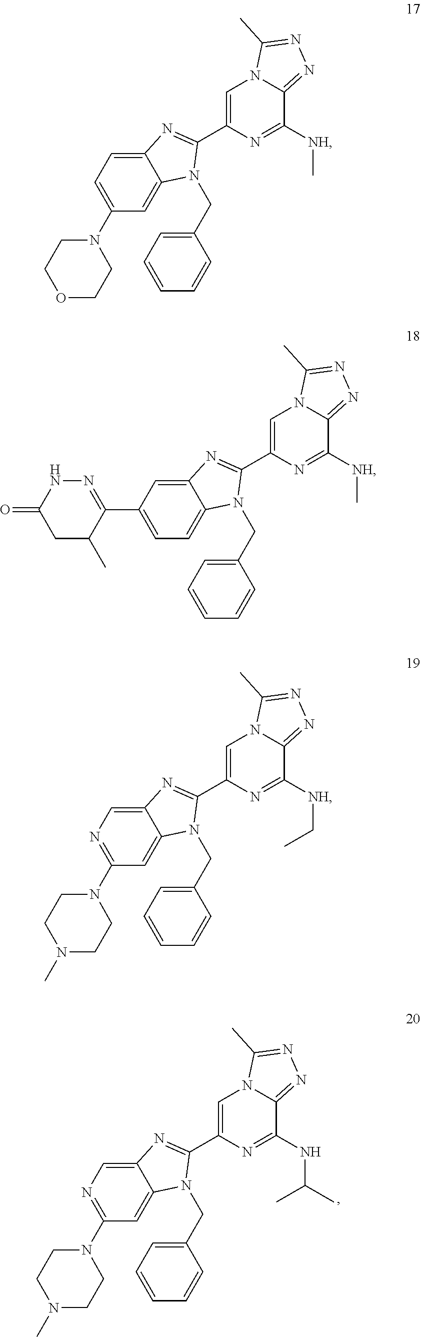 [SMILES: CNc1nc(-c2nc3ccc(N4CCOCC4)cc3n2Cc2ccccc2)cn2c(C)nnc12 ; CNc1nc(-c2nc3cc(C4=NNC(=O)CC4C)ccc3n2Cc2ccccc2)cn2c(C)nnc12 ; CCNc1nc(-c2nc3cnc(N4CCN(C)CC4)cc3n2Cc2ccccc2)cn2c(C)nnc12 ; Cc1nnc2c(NC(C)C)nc(-c3nc4cnc(N5CCN(C)CC5)cc4n3Cc3ccccc3)cn12]

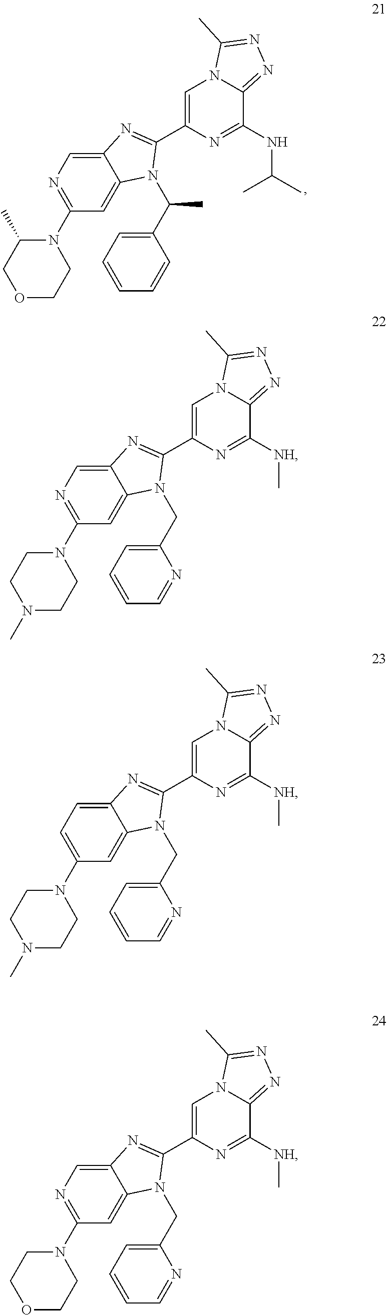 [SMILES: Cc1nnc2c(NC(C)C)nc(-c3nc4cnc(N5CCOC[C@@H]5C)cc4n3[C@@H](C)c3ccccc3)cn12 ; CNc1nc(-c2nc3cnc(N4CCN(C)CC4)cc3n2Cc2ccccn2)cn2c(C)nnc12 ; CNc1nc(-c2nc3ccc(N4CCN(C)CC4)cc3n2Cc2ccccn2)cn2c(C)nnc12 ; CNc1nc(-c2nc3cnc(N4CCOCC4)cc3n2Cc2ccccn2)cn2c(C)nnc12]

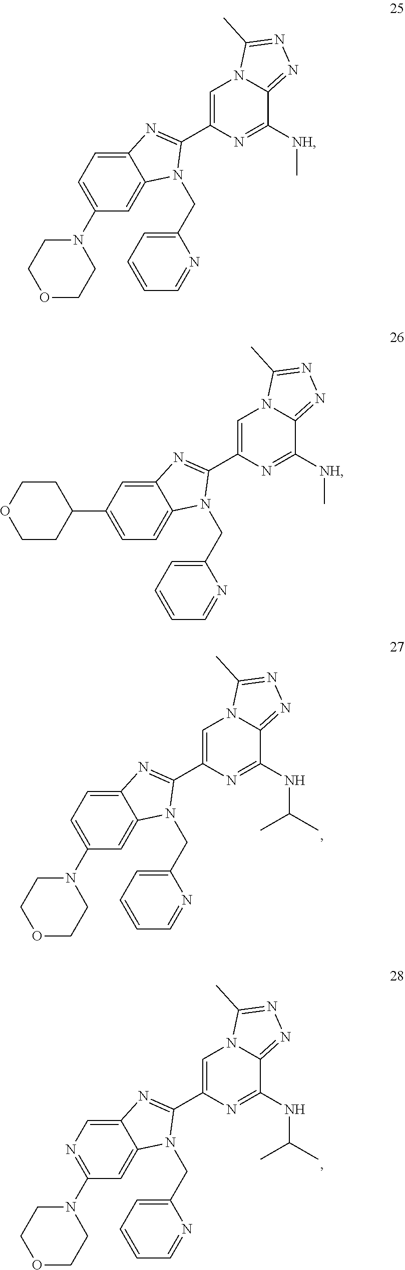

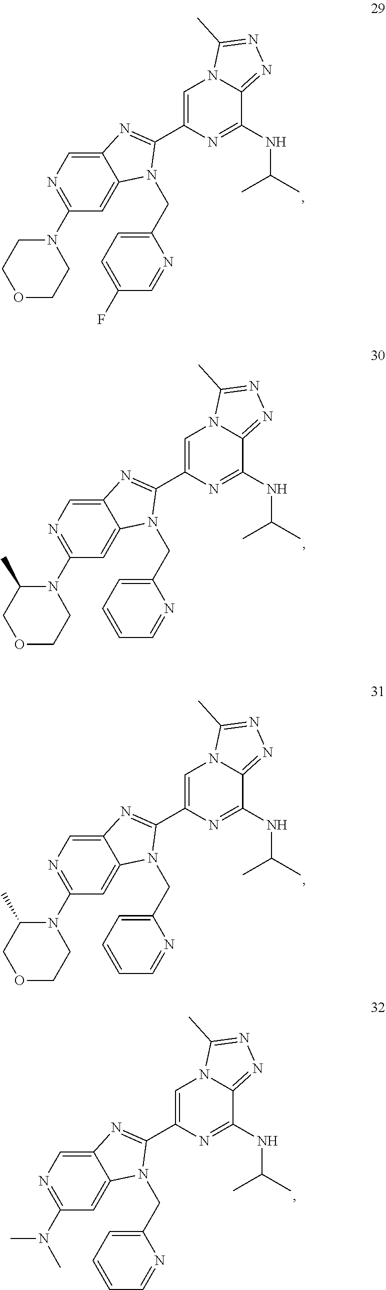

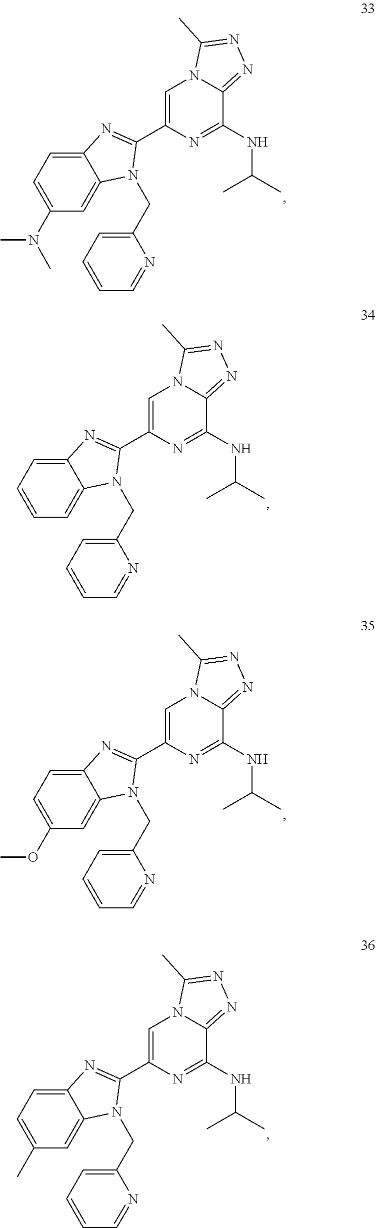

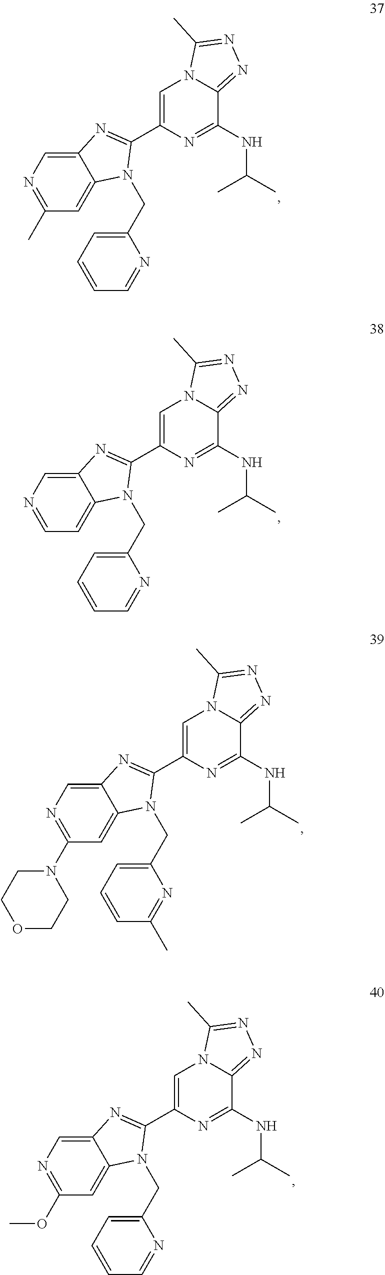

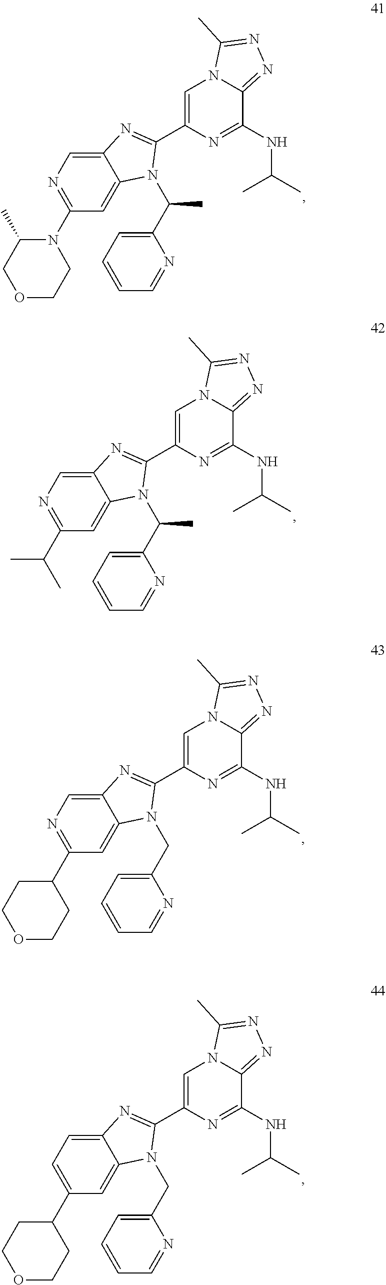

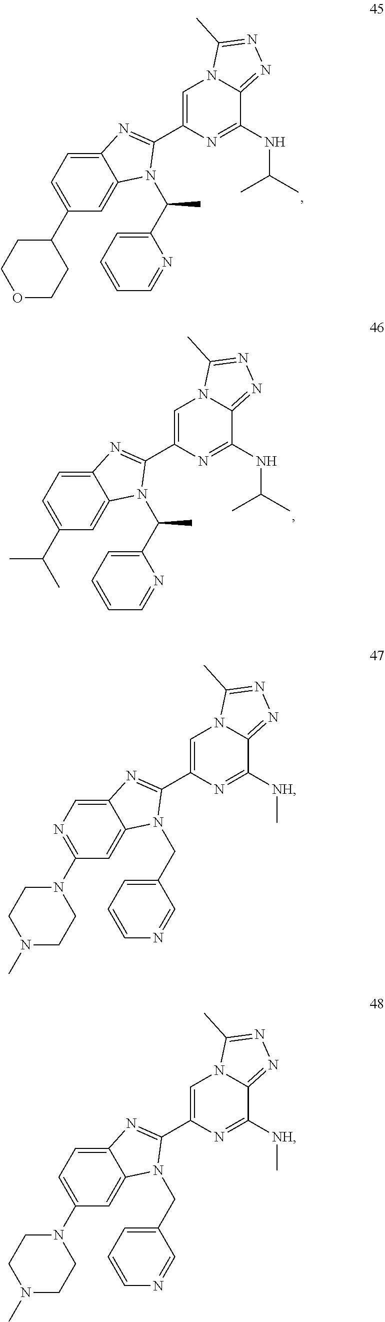

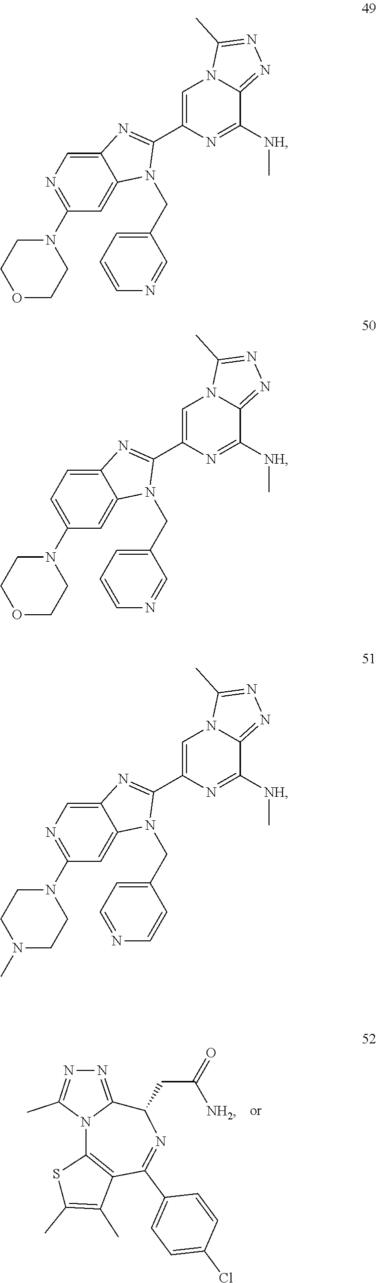

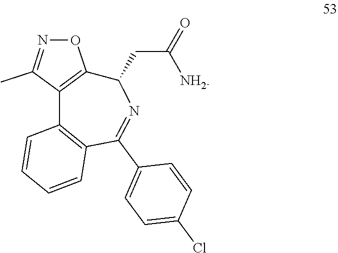 [SMILES: Cc1noc2c1-c1ccccc1C(c1ccc(Cl)cc1)=N[C@H]2CC(N)=O]

11. A kit comprising a pharmaceutical composition comprising volasertib, or a pharmaceutically acceptable salt thereof or a hydrate thereof, and another pharmaceutical composition comprising a BET inhibitor, or a pharmaceutically acceptable salt thereof or a hydrate thereof.

12. (canceled)

13. (canceled)
